(19) United States
(12) Patent Application Publication
(10) Pub. No.: US 2011/0112053 A1
(43) Pub. Date: May 12, 2011

(54) PHARMACOLOGICAL TARGETING OF VASCULAR MALFORMATIONS

(75) Inventors: Dean Li, Salt Lake City, UT (US); Kevin Whitehead, Salt Lake City, UT (US); Aubrey Chan, Salt Lake City, UT (US); Nyall London, West Valley, UT (US); Sutip Navankasattusas, Salt Lake City, UT (US)

(73) Assignee: University of Utah Research Foundation, Salt Lake City, UT (US)

(21) Appl. No.: 12/937,336

(22) PCT Filed: Apr. 16, 2009
(86) PCT No.: PCT/US09/40821

§ 371 Date: Dec. 22, 2010

Related U.S. Application Data
(60) Provisional application No. 61/045,446, filed on Apr. 16, 2008.

(51) Int. Cl. A61K 31/675; A61K 31/366; A61K 31/663; A61K 31/4164; A61K 31/216; A61K 31/4409; A61K 31/5513; A61P 25/08; A61P 9/00; A61P 9/10

(52) U.S. Cl. 514/89; 514/460; 514/108; 514/94; 514/400; 514/510; 514/352; 514/218

(57) ABSTRACT

Disclosed herein are compositions and methods for decreasing vascular permeability in a blood vessel and treating or preventing conditions associated with defects or injuries of vascular endothelium. For example, the disclosed compositions and methods can be used to treat a vascular dysplasia such as cerebral cavernous malformation (CCM). These methods relate generally to the use of compositions that inhibit RhoA GTPase levels or activity, such as inhibitors of 3-hydroxy-3-methylglutaryl-coenzyme A (HMG-CoA) reductase.

### CROSS-REFERENCE TO RELATED APPLICATIONS

This application claims benefit of U.S. Provisional Application No. 61/045,446, filed Apr. 16, 2009, which is hereby incorporated herein by reference in its entirety.

### STATEMENT REGARDING FEDERALLY SPONSORED RESEARCH

This invention was made with government support under Grants R01-HL068873, R01-HL077671, and K08-HL079095 awarded by the National Institutes of Health. The government has certain rights in the invention.

### BACKGROUND

Cerebral cavernous malformations (CCM) are common vascular malformations that affect the systemic and central nervous system (CNS) vasculature with a prevalence of 1:200-250 people (O. Del Curling, Jr., D. L. Kelly, Jr., A. D. Elster, T. E. Craven. 1991. J Neurosurg 75, 702; P. Otten, G. P. Pizzolato, B. Rilliet, J. Berney. 1989. Neurochirurgie 35, 82; J. R. Robinson, I. A. Awad, J. R. Little. 1991. J Neurosurg 75, 709; M. W. Vernooij et al. 2007. N Engl J Med 357, 1821) in unselected populations. Cavernous malformations consist of enlarged microvascular channels lined by a single layer of endothelium without underlying smooth muscle support. Those who harbor these vascular lesions are subject to an unpredictable risk of hemorrhage for which no pharmacologic therapy currently exists (J. L. Moriarity et al. 1999. Neurosurgery 44, 1166; T. Hasegawa et al. 2002. Neurosurgery 50, 1190). Even before an overt hemorrhage, all lesions are surrounded by hemosiderin, the iron laden deposits that result from extravascular blood. These iron deposits that result from vascular leakage can be sensitively detected by MRI (P. M. Chappell, G. K. Steinberg, M. P. Marks. 1992. Radiology 183:719) and suggest abnormal endothelial barrier function (R. E. Clatterbuck, C. G. Eberhart, B. J. Crain, D. Rigamonti. 2001. J Neurol Neurosurg Psychiatry 71:188). Although lesions have been described in a variety of vascular beds (I. Toldo, P. Drigo, I. Mammi, V. Marini, C. Carollo. 2009. 71(2):167-71), clinical manifestations are most common in the CNS. Depending on the location of lesions, the consequences of leak and hemorrhage can be stroke, seizure, or even death. Thus, needed are compositions and methods for treating or preventing conditions associated with defects or injuries of vascular endothelium.

### BRIEF SUMMARY

In accordance with the purpose of this invention, as embodied and broadly described herein, this invention relates to compositions and methods for decreasing vascular permeability in a blood vessel and treating or preventing conditions associated with defects or injuries of vascular endothelium, such as edema in a subject, comprising administering to the subject a RhoA GTPase inhibitor.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows Ccm2 (also known as OSM (osmosensing scaffold for MEKK3)) is required for circulation. FIG. 1A shows whole mount confocal immunofluorescent micrographs of littermate embryos at E8.5 stained for CD31 (PECAM). The vasculature is abnormal in the gene trap Ccm2^{tr/tr} homozygote (right). FIG. 1B shows higher magnification of the first branchial arch arteries (BAA1) and dorsal aorta (DA). See diagrams below for orientation. The wild type vessels have a uniform caliber (double arrows) capable of supporting flow. Ccm2-mutant vessels (single arrows) are narrower at BAA1 and adjacent portions of the dorsal aorta than wild-type vessels (double arrows).

FIG. 1C shows fetal ultrasound demonstrating no flow in a Ccm2^{tr/tr} embryo (bottom row) at E8.5 despite normal frequency of cardiac contractions (middle panel). Digital subtraction (right panels) was used to highlight moving blood in vessels of the wild type embryo. Blood flow was seen in wild type littermates (top row). See diagrams (left panels) for orientation; DA, dorsal aorta, YS, yolk sac. FIG. 1D shows ink injection into the cardiac ventricle. Ink passage through the branchial arch arteries at E9.5 was observed in a wild-type embryo (top) but no anterograde passage through the branchial arches (arrow) into the aorta was observed in a Ccm2^{tr/tr} littermate (bottom). Scale bars: 500 μm in A, C and D, and 200 μm in B. Results are representative of multiple (minimum eight) paired observations.

FIG. 2 shows vascular defects in mutant mice are endothelial autonomous. FIG. 2A shows whole-mount immunofluorescence for CD31 (PECAM) demonstrating normal, uniform caliber branchial arch arteries and aortae in all Ccm2^{fl/-} embryos except the endothelial (Tie2-CRE) mutant, which has an irregular, narrow lumen (arrowheads). Cartoons at the bottom are provided for orientation. BAA1, first branchial arch artery; BAA2, second branchial arch artery; VA, ventral aorta (or aortic sac); DA, dorsal aorta. FIG. 2B are paraffin sections taken at E9.0 and stained for CD31 showing the narrow branchial arch arteries in mutant embryos. In contrast to the wild type embryo (left panel) the first branchial arch artery (arrowhead) is similarly narrowed and irregular in both the complete knockout (Ccm^{tr/tr}, middle panel) and the endothelial mutant (Ccm^{fl/-};Tg(Tie2-CRE), right panel) embryos. Scale bars: 200 μm. Results are representative of multiple (minimum seven) independent observations.

FIG. 3 shows CCM2 is required for endothelial tube morphogenesis. FIG. 3A shows real-time quantitative RT-PCR demonstrating that CCM2 siRNA reduces the level of CCM2 transcripts in human dermal microvascular endothelial cells (HMVEC) and human umbilical vein endothelial cells (HMVEC) by 80%. Transcripts were normalized to GAPDH expression. FIG. 3B shows treatment with CCM2 siRNA significantly reduces tube formation of HUVECs in three-dimensional cultures in collagen as shown by staining with toluidine blue. Two separate control siRNAs (luciferase siRNA, or a nonsense control siRNA) do not affect endothelial tube formation. FIG. 3C shows time-lapse photography of tube development in endothelial cells treated with CCM2 siRNA compared with luciferase siRNAs. Arrows denote organization of lumenized structures from vacuole precursors. FIG. 3D shows quantification of lumen and vacuole development over time in HUVECs treated with CCM2 siRNA as compared to a luciferase (Luc) siRNA control. Five fields were analyzed for each data point. FIG. 3E shows quantification of lumen numbers at 24 h in HUVECs treated with CCM2 siRNA compared to luciferase or random siRNA controls. EC, endothelial cell; hpf, high power field. Three fields were analyzed for each siRNA. FIG. 3F shows CCM2 levels assayed by RT-PCR in control HUVECs undergoing tube formation at various stages of the vacuole and lumen formation assay. FIG. 3G shows quantification of filopodial length in HUVECs treated with CCM2 siRNA compared to a luciferase siRNA control. Ten fields were analyzed for each siRNA. FIG. 3H shows haptotactic migration of HMVECs to fibronectin in CCM2-depleted cells versus random siRNA control-treated cells. Analysis was performed on twelve control and eight CCM2 siRNA fields. Scale bars, 100 mm. Values are means ±s.e.m., except in Figure E, where values are means±s.d.

FIG. 4 shows CCM2 deficiency alters the endothelial cytoskeletal architecture and cell-cell interactions via activation of the small GTPase RHOA. FIG. 4A shows confocal immunofluorescent visualization of cellular cytoskeleton (actin fibers) and cell junctions (b-catenin) in HMVECs treated with CCM2 or random control siRNA. Results are representative of three independent experiments. FIG. 4B shows endothelial monolayer permeability to HRP in HMVECs treated with CCM2 or random control siRNA, as determined by absorbance at 490 nm (A490). FIG. 4C shows transendothelial resistance in CCM2-depleted HMVECs compared to control-treated cells. FIG. 4D shows GTPase pulldown assays for GTP-bound (active) RHOA, RAC1 and CDC42 in control and CCM2-depleted cells. Results are representative of three independent experiments. FIG. 4E shows immunoprecipitation assays for CCM2 binding to Rho family GTPases. Myc, construct with myc epitope tag; Vec, empty vector control; V5, construct with V5 epitope tag; IP, immunoprecipitation; Anti, antibody to the indicated protein. Results are representative of three independent experiments. FIG. 4F shows fluorescent phalloidin staining for actin stress fibers in HMVECs after treatment with inhibitors of Rho signaling. Results are representative of three independent experiments. FIG. 4G shows time course of transendothelial electrical resistance in CCM2-depleted HMVECs compared to cells treated with either C3-transferase or control. FIG. 4H shows immunoblot analyses of phosphorylated (active) MAP kinases and the JNK upstream kinases MKK4 and MKK7. p-Ab, phosphorylated kinase; t-Ab, total kinase. Results are representative of three independent experiments. FIG. 4I shows immunoblot analysis of cell lysates for phosphorylated and total JNK after treatment with the Rho-kinase inhibitor Y-27632. Results are representative of three independent experiments. Scale bars, 50 mm. Values are means±s.e.m. For b, c and g, a minimum of three independent experiments were performed.

FIG. 5 shows heterozygous Ccm2^{+/tr} mice have permeability defects that can be rescued by treatment with simvastatin. FIG. 5A shows spectrophotometric quantification of Evans blue extravasation in the Miles assay of dermal permeability in Ccm2^{+/tr} versus Ccm2^{+/+} mice across a range of doses of VEGF compared to saline control. Five mice were studied for each genotype. FIG. 5B shows quantification of dermal permeability in mice with endothelial specific heterozygosity for Ccm2 (Ccm2^{fl/+};Tg(Tie2-Cre)) compared to mice with both Ccm2 alleles intact (Ccm2^{fl/+}) and mice with complete Ccm2 heterozygosity (Ccm2^{fl/-}). Five Ccm2^{fl/+} mice, nine Ccm2^{fl/-} mice and ten Ccm2^{fl/+};Tg(Tie2-Cre) mice were studied. FIG. 5C shows phalloidin staining for cellular actin fibers after treatment with carrier or simvastatin. Results are representative of three independent experiments. FIG. 5D shows haptotactic migration of HMVECs to fibronectin after treatment with CCM2 or random control siRNA and treatment with either simvastatin or ethanol carrier. A minimum of three independent experiments were performed. FIG. 5E shows immunoblot for phosphorylated and total JNK in HMVECs treated with CCM2 or random control siRNA and treated with either simvastatin or ethanol carrier. Results are representative of three independent experiments. FIG. 5F shows quantification of Evans blue extravasation in the Miles assay in response to saline or VEGF after pretreatment with simvastatin or ethanol carrier. For both genotypes, three mice were used with control treatment and four mice were used with simvastatin treatment. Scale bars, 100 mm. Values are means±s.e.m.

FIG. 6 shows gene trap mutation results in loss of Ccm2 expression and angiogenesis defects. FIG. 6A shows the genomic structure of wild type Ccm2 is disrupted by insertion of the gene trap vector within exon 6, resulting in the loss of 45 nucleotides of wild type genomic DNA. The location of genotype primers is demonstrated. FIG. 6B shows the results of PCR genotyping for the three possible genotypes. FIG. 6C shows real-time quantitative RT PCR with primers in exons 8 and 9 for Ccm2 message in total RNA derived from Ccm2^{tr/tr} embryos. The quantity of Ccm2 cDNA was normalized to Gapdh (values+/−s.d.). FIG. 6D shows the aorta of the embryo (arrows) caudal to the heart and venous inflow in wild type and mutant embryos. The aortae of the mutant enlarge by E9.0 (lower right). FIG. 6E shows development of the first branchial artery in mice lacking Ccm2. A cord of endothelial cells is present at E8.0 in both wild type and mutant embryos (arrows, upper panels). The mutant has endothelial cells without proper lumen at E9.0 (arrows, lower panels). FIG. 6F shows angiogenesis defects involve the intersomitic arteries in mice lacking Ccm2. Intersomitic artery sprouts (arrows) are broad and irregular in mutant embryos. An abnormal, direct connection between the cardinal vein (arrowheads) and dorsal aorta (arrows) is seen in a mutant E9.0 embryo (lower right). Scale bars: 100 μm.

FIG. 7 shows conditional targeting of Ccm2. The three alleles of Ccm2 that result from the disclosed targeting strategy are shown. FIG. 7A shows wild type Ccm2 has 10 exons, the final 9 of which are shown. FIG. 7B shows the conditional (floxed) allele includes LoxP sites that flank exons 3 through 10 of Ccm2. The floxed allele can be detected with primers W and X, or can be recognized by the upward shift in band size with primers Y and Z relative to wild type, owing to the insertion of the short LoxP sequence. FIG. 7C shows exposure of the conditional (floxed) allele to CRE recombinase results in deletion of all sequence between the LoxP sites, including exons 3-10 of Ccm2. The mutant allele can be detected with primers X and Y. FIG. 7D shows PCR genotyping results are shown for all 6 possible combinations. FIG. 7E shows confocal immunofluorescence (CD31 antibody) of branchial arch arteries (arrows) and aorta in an embryo homozygous for a germline recombined allele of Ccm2 compared to a wild type littermate. Scale bars: 100 μm. FIG. 7F shows X-gal staining of embryos containing LacZ reporter allele and tissue specific Cre drivers as specified. The branchial artery endothelium is indicated (arrows). Scale bars: 500 μm. FIG. 6G shows PCR for the recombined allele in embryos (primers X-Y, “RECOMB”). Primers Y-Z also define the status of the wild type (“WT”) and floxed (“FL”) alleles. The appearance of PCR product for recombined allele in Ccm2^{fl/+} embryos (arrows) indicates Cre-mediated recombination for each of the tissue specific drivers.

FIG. 8 illustrates the cholesterol synthesis pathway involved in Rho posttranslational modification.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and discussed and a number of modifications that can be made to a number of molecules including the compound are discussed, each and every combination and permutation of compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents.

It must be noted that as used herein and in the appended claims, the singular forms “a,” “an,” and “the” include plural reference unless the context clearly dictates otherwise. Thus, for example, reference to “a compound” includes a plurality of such compounds, reference to “the compound” is a reference to one or more compounds and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Activities” of a protein include, for example, transcription, translation, intracellular translocation, secretion, phosphorylation by kinases, cleavage by proteases, homophilic and heterophilic binding to other proteins, ubiquitination.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

As disclosed herein, Ccm2 (also known as Osm) is required for the first essential angiogenic event during development, the formation of the first branchial arch artery. Moreover, vascular defects associated with Ccm2 mutations are endothelial autonomous. Cultured endothelial cells with reduced Ccm2 expression have intrinsic impairment of lumen formation and bear many hallmarks of RhoA GTPase activation, such as actin stress fiber formation and activation of the stress activated kinase JNK. Inhibitors of Rho can reverse the cytoskeletal changes and JNK hyperphosphorylation in cells. Decreased endothelial barrier function is observed in cultured endothelial cells and in mice with heterozygous mutations of Ccm2 that genocopy human CCM. Impaired barrier function can also be rescued in vivo by pre-treatment with simvastatin, a known indirect inhibitor of Rho GTPases. This work indicates a pathologic role for increased Rho signaling in the endothelium of CCM and targeted pharmacologic therapies to address vascular defects in this common condition. Thus, disclosed herein are compositions and methods relating to the use of RhoA GTPase inhibitors.

For example, disclosed herein is a RhoA GTPase inhibitor that inhibits the expression, activation, or down-stream signaling of RhoA. The RhoA GTPase inhibitor can act directly on RhoA or affect molecules or proteins that naturally regulate RhoA activation, expression, or signaling. Thus, the RhoA GTPase inhibitor can be a composition, such as a functional nucleic acid, that inhibits RhoA levels or expression. The RhoA GTPase inhibitor can be a molecule, such as an antibody or soluble receptor/ligand that inhibits the binding of RhoA to other molecules or proteins, such as for example, ROCK1, DIAPH1, GTP/GDP.

Rho-associated, coiled-coil containing protein kinase 1 (ROCK1) is activated when bound to the GTP-bound form of Rho GTPase. This protein is thus a downstream effector of Rho. It phosphorylates and activates LIM kinase, which in turn, phosphorylates cofilin, inhibiting its actin-depolymerizing activity. Thus, ROCK1 contributes to actin-stability. Thus, in some aspects of the method, the RhoA GTPase inhibitor inhibits ROCK1 activity, activation, and/or expression

As disclosed herein CCM2 deletion results in RhoA activation and downstream activation of stress activated kinsase JNK. C-Jun N-terminal kinases (JNKs), originally identified as kinases that bind and phosphosphorylate c-Jun on Ser63 and Ser73 within its transcriptional activation domain, are mitogen-activated protein kinases which are responsive to stress stimuli, such as cytokines, ultraviolet irradiation, heat shock, and osmotic shock, and are involved in T cell differentiation and apoptosis. The c-Jun N-terminal kinases consist of ten isoforms deriving from the three genes JNK1, JNK2 and JNK3. JNK1 is involved in apoptosis, neurodegeneration, cell differentiation and proliferation, inflammatory conditions and cytokine production mediated by AP-1 (Activation Protein 1) such as RANTES, IL-8 and GM-CSF. JNKs can associate with scaffold proteins JNK Interacting Proteins as well as their upstream kinases JNKK1 and JNKK2 following their activation. JNK, by phosphorylation, modifies the activity of numerous proteins that reside at the mitochondria or act in the nucleus. This way, JNK activity regulates several important cellular functions. Inflammatory signals, changes in levels of reactive oxygen species, Ultraviolet radiation, protein synthesis inhibitors, and a variety of stress stimuli can activate JNK. One way this activation may occur is through disruption of the conformation of sensitive protein phosphatase enzymes; specific phosphatases normally inhibit the activity of JNK itself and the activity of proteins linked to JNK activation. Thus, in some aspects of the method, the RhoA GTPase inhibitor inhibits JNK activity, activation, and/or expression. In some aspects, the RhoA GTPase inhibitor inhibits a kinase upstream of JNK. Thus, in some aspects of the method, the RhoA GTPase inhibitor inhibits MKK4 or MKK7.

1. RhoA Prenylation

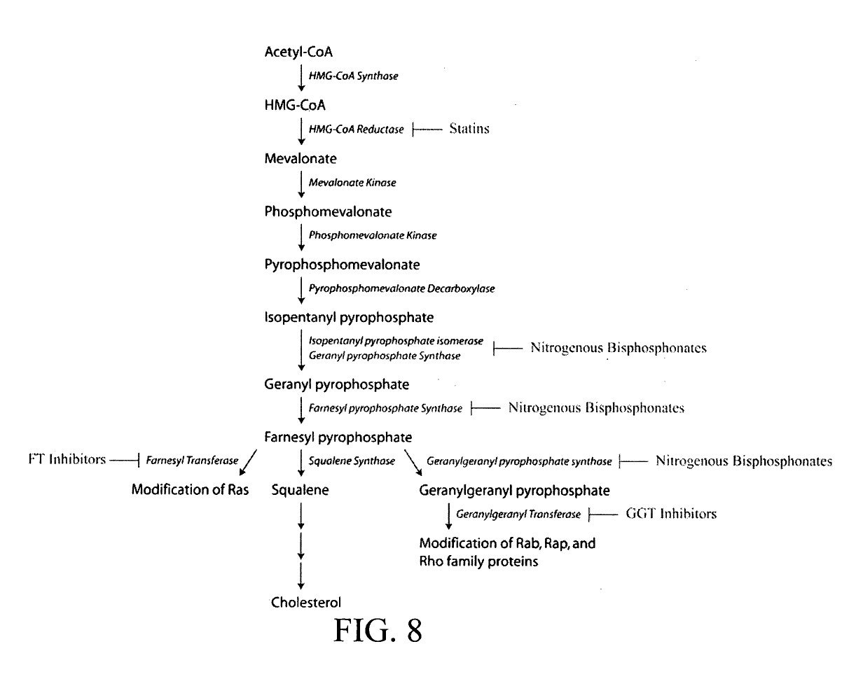

The RhoA GTPase inhibitor can inhibit posttranslational modification of RhoA. Thus, the RhoA GTPase inhibitor can be an inhibitor of RhoA isoprenylation. Important soprenoid intermediates are produced as part of the cholesterol biosynthetic pathway during L-mevalonic acid synthesis. These include farnesylpyrophosphate (FPP) and geranylgeranylpyrophosphate (GGPP). These intermediates serve as important lipid attachments for the posttranslational modification of a variety of cell-signaling proteins. Protein isoprenylation permits the covalent attachment, subcellular localization, and intracellular trafficking of membrane-associated proteins. Members of the Ras and Rho GTPase family are major substrates for posttranslational modification by isoprenylation. FIG. 8 illustrates the enzymes and intermediates involved in the cholesterol synthesis pathway that result in RhoA isoprenylation. Thus, the RhoA GTPase inhibitor of the disclosed methods can be an inhibitor of one or more of the enzymes in the cholesterol synthesis pathway that result in RhoA isoprenylation.

i. HMG-CoA Reductase Inhibitors

Thus, in some aspects of the method, the RhoA GTPase inhibitor is an inhibitor of 3-hydroxy-3-methylglutaryl-coenzyme A (HMG-CoA) reductase. HMG-CoA reductase (HMGR) is the rate controlling enzyme (EC 1.1.1.88) of the mevalonate pathway, the metabolic pathway that produces cholesterol and other isoprenoids. This enzyme is anchored in the membrane of the endoplasmic reticulum.

Drugs which inhibit HMG-CoA reductase, known collectively as HMG-CoA reductase inhibitors (or “statins”), include lovastatin, fluvastatin, atorvastatin, pravastatin, and simvastatin. Vytorin is drug that combines the use simvastatin and ezetimibe, which blocks the formation of cholesterol by the body, along with the absorption of cholesterol in the intestines.

Thus, the RhoA GTPase inhibitor can be a statin molecule. For example, the statin molecule can be selected from the group consisting of Lovastatin, Pravastatin, Simvastatin, Fluvastatin, Atorvastatin, or Cerivastatin. In some aspects of the method, the statin molecule is in a lactone form prior to administration. In some aspects of the method, the HMG CoA reductase inhibitor is administered orally. In some aspects of the method, the HMG CoA reductase inhibitor is administered locally to the vascular dysplasia.

U.S. Pat. No. 4,231,938, U.S. Pat. No. 5,712,130, and U.S. Pat. No. 7,052,886 are hereby incorporated herein by reference for the teachings of the structure of and methods of making and using Lovastatin.

U.S. Pat. No. 4,346,227, U.S. Pat. No. 6,740,775, U.S. Pat. No. 7,078,558 are hereby incorporated herein by reference for the teachings of the structure of and methods of making and using Pravastatin.

U.S. Pat. No. 4,444,784, U.S. Pat. No. 6,576,775, U.S. Pat. No. 6,825,362 are hereby incorporated herein by reference for the teachings of the structure of and methods of making and using Simvastatin.

U.S. Pat. No. 5,354,772 and U.S. Pat. No. 6,858,643 are hereby incorporated herein by reference for the teachings of the structure of and methods of making and using Fluvastatin. U.S. Pat. No. 4,739,073 is hereby incorporated herein by reference for the teachings of Fluvastatin as racemate as well as the single enantiomers.

U.S. Pat. No. 4,681,893 and U.S. Pat. No. 5,273,995 are hereby incorporated herein by reference for the teachings of the structure of and methods of making and using Atorvastatin.

U.S. Pat. No. 5,006,530 is hereby incorporated herein by reference for the teachings of the structure of and methods of making and using Cerivastatin.

In some aspects of the method, the RhoA GTPase inhibitor enhances degradation of HMG CoA reductase rather than inhibiting its enzymatic activity.

ii. Farnesyl Diphosphate Synthase Inhibitors

In some aspects of the method, the RhoA GTPase inhibitor is an inhibitor of farnesyl diphosphate synthase (FPPS). Thus, in some aspects of the method, the RhoA GTPase inhibitor is a nitrogen-containing bisphosphonate. Non-limiting examples of nitrogen-containing bisphosphonate include Pamidronate, Neridronate, Olpadronate, Alendronate, Ibandronate, Risedronate, and Zoledronate. Thus, the RhoA GTPase inhibitor can have the structure:

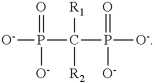

where R₁ is OH and wherein R₂ is

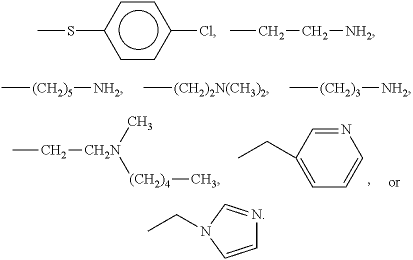

Nitrogenous bisphosphonates act on bone metabolism by binding and blocking the enzyme farnesyl diphosphate synthase (FPPS) in the HMG-CoA reductase pathway (also known as the mevalonate pathway). In enzymology, a Z-farnesyl diphosphate synthase (EC 2.5.1.68) is an enzyme that catalyzes the chemical reaction:

geranyl diphosphate+isopentenyl diphosphate [symbol] diphosphate+(2Z,6E)-farnesyl diphosphate

Thus, the two substrates of this enzyme are geranyl diphosphate and isopentenyl diphosphate, whereas its two products are diphosphate and (2Z,6E)-farnesyl diphosphate.

This enzyme belongs to the family of transferases, specifically those transferring aryl or alkyl groups other than methyl groups. The systematic name of this enzyme class is geranyl-diphosphate:isopentenyl-diphosphate geranylcistransferase. This enzyme is also called (Z)-farnesyl diphosphate synthase.

iii. Geranylgeranyl Transferase Inhibitors

Thus, in some aspects of the method, the RhoA GTPase inhibitor is an inhibitor of Geranylgeranyl Transferase. Thus, in some aspects of the method, the RhoA GTPase inhibitor is GGTI-2133 or GGTI-298.

GGTI-2133 is a cell-permeable non-thiol peptidomimetic that acts as a potent and selective inhibitor of geranylgeranyltransferase I (GGTase I; IC₅₀=38 nM) with a 140-fold selectivity over farnesyltransferase (FTase; IC₅₀=5.4 μM). Thus, in some aspects of the method, the RhoA GTPase inhibitor is N-[[4-(Imidazol-4-yl)methylamino]-2-(1-naphthyl)benzoyl]leucine trifluoroacetate salt. Thus, in some aspects of the method, the RhoA GTPase inhibitor has the structure:

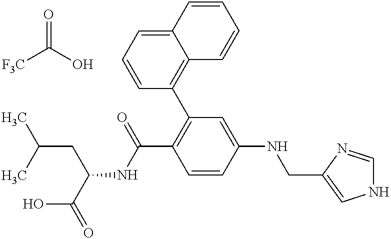

Thus, in some aspects of the method, the RhoA GTPase inhibitor is N-[[4-(2-(R)-Amino-3-mercaptopropyl)amino]-2-naphthylbenzoyl]leucine methyl ester trifluoroacetate salt. Thus, in some aspects of the method, the RhoA GTPase inhibitor has the structure:

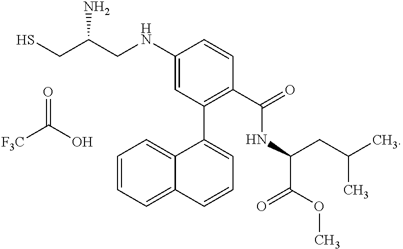

iv. Prenyl Transferase Inhibitors

Thus, in some aspects of the method, the RhoA GTPase inhibitor is an inhibitor of Prenyl Transferase. Prenyltransferases are a class of enzymes that transfer allylic prenyl groups to acceptor molecules. Prenyl transferases commonly refer to prenyl diphosphate synthases. Prenyltransferases are commonly divided into two classes, cis (or Z) and trans (or E), depending upon the stereo chemistry of the resulting products. Examples of trans-prenyltransferases include dimethylallyltranstransferase, and geranylgeranyl pyrophosphate synthase. Cis-prenyltransferases include dehydrodolichol diphosphate synthase (involved in the production of a precursor to dolichol). U.S. Pat. No. 6,376,468, U.S. Pat. No. 5,767,274, and U.S. Pat. No. 6,586,461 are hereby incorporated by reference herein for their teachings of Prenyl Transferase Inhibitors and how to make and use same.

v. Rho Kinase (ROCK1) Inhibitor

In some aspects of the method, the RhoA GTPase inhibitor is an inhibitor of Rho Kinase (ROCK1). Thus, in some aspects of the method, the RhoA GTPase inhibitor is (R)-(+)-trans-N-(4-Pyridyl)-4-(1-aminoethyl)-cyclohexanecarboxamide (Y-27632). Thus, in some aspects of the method, the RhoA GTPase inhibitor has the structure:

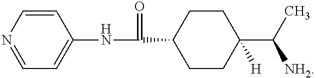

Thus, in some aspects of the method, the RhoA GTPase inhibitor is Fasudil (HA1077). Thus, in some aspects of the method, the RhoA GTPase inhibitor is 5-(1,4-diazepane-1-sulfonyl)isoquinoline. Thus, in some aspects of the method, the RhoA GTPase inhibitor has the structure:

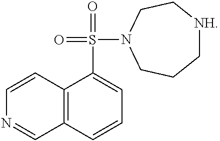

Thus, in some aspects of the method, the RhoA GTPase inhibitor is (S)-(+)-2-Methyl-1-[(4-methyl-5-isoquinolinyl)sulfonyl]-hexahydro-1H-1,4-diazepine dihydrochloride (H 1152). Thus, in some aspects of the method, the RhoA GTPase inhibitor has the structure:

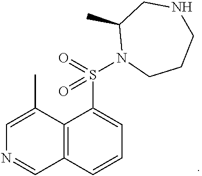

Thus, in some aspects of the method, the RhoA GTPase inhibitor is Hydroxyfasudil (HA 1100). 1-[(1,2-Dihydro-1-oxo-5-isoquinolinyl)sulfonyl]hexahydro-1H-1,4-diazepine. Thus, in some aspects of the method, the RhoA GTPase inhibitor has the structure:

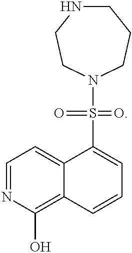

Thus, in some aspects of the method, the RhoA GTPase inhibitor is (+)-(R)-4-(1-aminoethyl)-N-(4-pyridyl) benzamide monohydrochloride (Wf-536).

2. Exoenzyme

The RhoA GTPase inhibitor can be an exoenzyme that ribosylates RhoA. For example, the exoenzyme C3 transferase is an ADP ribosyl transferase that selectively ribosylates RhoA, RhoB and RhoC proteins on asparagine residue 41, rendering them inactive. It has extremely low affinity for other members of the Rho family such as Cdc42 and Rac1 and does therefore not affect these GTPases. Hence, C3 transferase is a very potent and useful reagent to specifically block RhoA/B/C signaling.

3. Soluble Ligand/Receptor

The RhoA GTPase inhibitor can be a molecule, such as an antibody or soluble receptor/ligand that inhibits the binding of RhoA to other molecules or proteins, such as for example, ROCK1, DIAPH1, GTP/GDP.

Thus, in some aspects, the RhoA GTPase inhibitor comprises a polypeptide fragment of ROCK1 capable of binding to RhoA. The binding of ROCK1 to RhoA requires amino acids 948-1014 of ROCK1. Thus, in some aspects the RhoA GTPase inhibitor comprises amino acids 948-1014 of SEQ ID NO: 17 or a fragment or conservative variant thereof capable of binding RhoA.

Thus, in some aspects, the RhoA GTPase inhibitor comprises a polypeptide fragment of DIAPH1 capable of binding to RhoA. The binding of DIAPH1 to Rho family GTPases requires amino acids 84-269 of DIAPH1 transcript variant 1, and amino acids 75-260 of DIAPH1 transcript variant 2. Thus, in some aspects the RhoA GTPase inhibitor comprises amino acids 84-269 of SEQ ID NO: 19 or a fragment or conservative variant thereof capable of binding RhoA. Thus, in some aspects the RhoA GTPase inhibitor comprises amino acids 75-260 of SEQ ID NO: 21 or a fragment or conservative variant thereof capable of binding RhoA.

Thus, in some aspects, the RhoA GTPase inhibitor comprises a polypeptide fragment of RhoA capable of binding to ROCK1, DIAPH1, and/or GTP/GDP. While no specific domain of RhoA is responsible for binding, amino acids 15-20, 34-35, 37, 59-60, 118, 120, and 161-162 are involved in binding based on studies of crystal structure. Thus, in some aspects the RhoA GTPase inhibitor comprises amino acids 15-162 of SEQ ID NO:15 or a fragment or conservative variant thereof capable of binding ROCK1, DIAPH1, and/or GTP/GDP. Thus, in some aspects the RhoA GTPase inhibitor comprises amino acids 15-20, 34-35, 37, 59-60, 118, 120, and 161-162 of SEQ ID NO: 15 or a fragment or conservative variant thereof capable of binding ROCK1, DIAPH1, and/or GTP/GDP.

i. Antibodies

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with RhoA such that RhoA is inhibited from interacting with ROCK1, DIAPH1, and/or GTP/GDP. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

If these approaches do not produce neutralizing antibodies, cells expressing cell surface localized versions of these proteins will be used to immunize mice, rats or other species. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding extracellular fragments of RhoA, ROCK1, DIAPH1, or GTP/GDP expressed as a fusion protein with human IgG1 or an epitope tag is injected into the host animal according to methods known in the art.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves RhoA, ROCK1, DIAPH1, or GTP/GDP as fusion proteins with a signal sequence fragment. The antigen is produced by inserting a gene fragment in-frame between the signal sequence and the RhoA, ROCK1, DIAPH1, or GTP/GDP nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies. The culture medium in which the hybridoma cells are cultured can then be assayed for the presence of monoclonal antibodies directed against RhoA, ROCK1, DIAPH1, or GTP/GDP. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art.

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

a. Whole Immunoglobulin

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

b. Antibody Fragments

The term “antibody” as used herein is meant to include intact molecules as well as fragments thereof, such as, for example, Fab and F(ab′)₂, which are capable of binding the epitopic determinant.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain RhoA, ROCK1, DIAPH1, or GTP/GDP binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity.

Also included within the meaning of “antibody or fragments thereof” are conjugates of antibody fragments and antigen binding proteins (single chain antibodies).

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

Alternatively, unprotected peptide segments are chemically linked where the bond formed between the peptide segments as a result of the chemical ligation is an unnatural (non-peptide) bond. This technique has been used to synthesize analogs of protein domains as well as large amounts of relatively pure proteins with full biological activity.

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with RhoA, ROCK1, DIAPH1, or GTP/GDP. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen.

Techniques can also be adapted for the production of single-chain antibodies specific to an antigenic protein of the present disclosure. In addition, methods can be adapted for the construction of F (ab) expression libraries to allow rapid and effective identification of monoclonal F (ab) fragments with the desired specificity for a protein or derivatives, fragments, analogs or homologs thereof. Antibody fragments that contain the idiotypes to a protein antigen may be produced by techniques known in the art including, but not limited to: (i) an F ((ab′))(2) fragment produced by pepsin digestion of an antibody molecule; (ii) an Fab fragment generated by reducing the disulfide bridges of an F ((ab′))(2) fragment; (iii) an F (ab) fragment generated by the treatment of the antibody molecule with papain and a reducing agent and (iv) F (v), fragments.

Methods for the production of single-chain antibodies are well known to those of skill in the art. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation. These Fvs lack the constant regions (Fc) present in the heavy and light chains of the native antibody.

c. Monovalent Antibodies

In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

d. Chimeric/Hybrid

In hybrid antibodies, one heavy and light chain pair is homologous to that found in an antibody raised against one antigen recognition feature, e.g., epitope, while the other heavy and light chain pair is homologous to a pair found in an antibody raised against another epitope. This results in the property of multi-functional valency, i.e., ability to bind at least two different epitopes simultaneously. As used herein, the term “hybrid antibody” refers to an antibody wherein each chain is separately homologous with reference to a mammalian antibody chain, but the combination represents a novel assembly so that two different antigens are recognized by the antibody. Such hybrids can be formed by fusion of hybridomas producing the respective component antibodies, or by recombinant techniques. Such hybrids may, of course, also be formed using chimeric chains.

e. Method of Making Antibodies Using Protein Chemistry

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonyl) chemistry. One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains. Alternatively, native chemical ligation of synthetic peptides can be utilized to synthetically construct large peptides or polypeptides from shorter peptide fragments. This method consists of a two step chemical reaction. The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site.

f. Human and Humanized

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge. Human antibodies can also be produced in phage display libraries.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a non-human antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

As used herein, the term “epitope” is meant to include any determinant capable of specific interaction with the anti-RhoA, anti-ROCK1, anti-DIAPH1, or anti-GTP/GDP antibodies disclosed. Epitopic determinants usually consist of chemically active surface groupings of molecules such as amino acids or sugar side chains and usually have specific three dimensional structural characteristics, as well as specific charge characteristics.

An “epitope tag” denotes a short peptide sequence unrelated to the function of the antibody or molecule that can be used for purification or crosslinking of the molecule with anti-epitope tag antibodies or other reagents.

By “specifically binds” is meant that an antibody recognizes and physically interacts with its cognate antigen (e.g., a RhoA, ROCK1, DIAPH1, or GTP/GDP peptide) and does not significantly recognize and interact with other antigens; such an antibody may be a polyclonal antibody or a monoclonal antibody, which are generated by techniques that are well known in the art.

The antibody can be bound to a substrate or labeled with a detectable moiety or both bound and labeled. The detectable moieties contemplated with the present compositions include fluorescent, enzymatic and radioactive markers.

ii. Nucleic Acids

There are a variety of molecules disclosed herein that are nucleic acid based. The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

b. Sequences

There are a variety of sequences related to the protein molecules involved in the signaling pathways disclosed herein. The sequences for the human analogs of these genes, as well as other anlogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c. Primers and Probes

Disclosed are compositions including primers and probes, which are capable of interacting with the disclosed nucleic acids. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

d. Cell Delivery Systems

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(A) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus.

As used herein, plasmid or viral vectors are agents that transport the disclosed nucleic acids into the cell without degradation and include a promoter yielding expression of the gene in the cells into which it is delivered. In some embodiments the promoters are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The benefit of the use of replication-defective adenoviruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites. Recombinant adenoviruses achieve gene transduction by binding to specific cell surface receptors, after which the virus is internalized by receptor-mediated endocytosis, in the same manner as wild type or replication-defective.

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses. These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(B) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue, the principles of which can be applied to targeting of other cells. These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

iii. Peptides

a. Protein Variants

Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place.

Substantial changes in function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deaminated to the corresponding glutamyl and asparyl residues. Alternatively, these residues are deaminated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains, acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level. Another way of calculating homology can be performed by published algorithms.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

b. Internalization Sequences

The provided polypeptide can further constitute a fusion protein or otherwise have additional N-terminal, C-terminal, or intermediate amino acid sequences, e.g., linkers or tags. “Linker”, as used herein, is an amino acid sequences or insertion that can be used to connect or separate two distinct polypeptides or polypeptide fragments, wherein the linker does not otherwise contribute to the essential function of the composition. A polypeptide provided herein, can have an amino acid linker comprising, for example, the amino acids GLS, ALS, or LLA. A “tag”, as used herein, refers to a distinct amino acid sequence that can be used to detect or purify the provided polypeptide, wherein the tag does not otherwise contribute to the essential function of the composition. The provided polypeptide can further have deleted N-terminal, C-terminal or intermediate amino acids that do not contribute to the essential activity of the polypeptide.

The disclosed composition can be linked to an internalization sequence or a protein transduction domain to effectively enter the cell. Recent studies have identified several cell penetrating peptides, including the TAT transactivation domain of the HIV virus, antennapedia, and transportan that can readily transport molecules and small peptides across the plasma membrane. More recently, polyarginine has shown an even greater efficiency of transporting peptides and proteins across the plasma, membrane making it an attractive tool for peptide mediated transport. Nonaarginine has been described as one of the most efficient polyarginine based protein transduction domains, with maximal uptake of significantly greater than TAT or antennapeadia. Peptide mediated cytotoxicity has also been shown to be less with polyarginine-based internalization sequences. Polyarginine (e.g., R₉) mediated membrane transport is facilitated through heparan sulfate proteoglycan binding and endocytic packaging. Once internalized, heparan is degraded by heparanases, releasing R₉ which leaks into the cytoplasm. Studies have recently shown that derivatives of polyarginine can deliver a full length p53 protein to oral cancer cells, suppressing their growth and metastasis, defining polyarginine as a potent cell penetrating peptide.

Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol). Any other internalization sequences now known or later identified can be combined with a peptide for use in the disclosed compositions and methods.

4. Functional Nucleic Acids

The RhoA GTPase inhibitor of the provided method can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of RhoA, ROCK1, or JNK1 or the genomic DNA of RhoA, ROCK1, or JNK1 or they can interact with the polypeptide RhoA, ROCK1, or JNK1. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d})less than or equal to 10⁻⁶, 10⁻⁸, 10⁻¹⁰, or 10⁻¹².

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, hairpin ribozymes, and tetrahymena ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate.

Similarly, eukaryotic EGS/RNAse P-directed cleavage of RNA can be utilized to cleave desired targets within eukaryotic cells.

Gene expression can also be effectively silenced in a highly specific manner through RNA interference (RNAi). This silencing was originally observed with the addition of double stranded RNA (dsRNA). In an ATP dependent step, the siRNAs become integrated into a multi-subunit protein complex, commonly known as the RNAi induced silencing complex (RISC), which guides the siRNAs to the target RNA sequence. At some point the siRNA duplex unwinds, and it appears that the antisense strand remains bound to RISC and directs degradation of the complementary mRNA sequence by a combination of endo and exonucleases. However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

Disclosed herein are any siRNA designed as described above based on the sequences for RhoA, ROCK1, or JNK1. For example, a nucleic acid sequence for RhoA is set forth in SEQ ID NO: 16. A nucleic sequence for ROCK1 is set forth in SEQ ID NO: 18. A nucleic sequence for DIAPH1 is set forth in SEQ ID NO: 20 and SEQ ID NO: 22. A nucleic sequence for JNK1 is set forth in SEQ ID NO: 24, SEQ ID NO: 26, SEQ ID NO: 28, and SEQ ID NO: 30.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

5. Carriers

The disclosed compositions can be combined, conjugated or coupled with or to carriers and other compositions to aid administration, delivery or other aspects of the compositions and their use. For convenience, such composition will be referred to herein as carriers. Carriers can, for example, be a small molecule, pharmaceutical drug, fatty acid, detectable marker, conjugating tag, nanoparticle, or enzyme.

The disclosed compositions can be used therapeutically in combination with a pharmaceutically acceptable carrier. By “pharmaceutically acceptable” is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

The carrier molecule can be covalently linked to the disclosed inhibitors. The carrier molecule can be linked to the amino terminal end of the disclosed peptides. The carrier molecule can be linked to the carboxy terminal end of the disclosed peptides. The carrier molecule can be linked to an amino acid within the disclosed peptides. The herein provided compositions can further comprise a linker connecting the carrier molecule and disclosed inhibitors. The disclosed inhibitors can also be conjugated to a coating molecule such as bovine serum albumin (BSA) that can be used to coat microparticles, nanoparticles of nanoshells with the inhibitors.

Protein crosslinkers that can be used to crosslink the carrier molecule to the inhibitors, such as the disclosed peptides, are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimidyl) suberate), SMPB (succinimidyl-4-(p-maleimidophenyl)butyrate), SULFO SMPB (sulfosuccinimidyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO SIAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimidyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent) (Methyl-p-hydroxybenzimidate hydrochloride, 98%), DMA (Dimethyladipimidate hydrochloride).

i. Nanoparticles, Microparticles, and Microbubbles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels.

Nanoparticles, such as, for example, silica nanoparticles, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nano crystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide.

For example, the disclosed compositions can be immobilized on silica nanoparticles (SNPs). SNPs have been widely used for biosensing and catalytic applications owing to their favorable surface area-to-volume ratio, straightforward manufacture and the possibility of attaching fluorescent labels, magnetic nanoparticles and semiconducting nanocrystals.

The nanoparticle can also be, for example, a heat generating nanoshell. As used herein, “nanoshell” is a nanoparticle having a discrete dielectric or semi-conducting core section surrounded by one or more conducting shell layers.

Targeting molecules can be attached to the disclosed compositions and/or carriers. For example, the targeting molecules can be antibodies or fragments thereof, ligands for specific receptors, or other proteins specifically binding to the surface of the cells to be targeted.

ii. Liposomes

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae.

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles.

Small ULVs can also be prepared by the ethanol injection technique and the ether injection technique. These methods involve the rapid injection of an organic solution of lipids into a buffer solution, which results in the rapid formation of unilamellar liposomes. Another detergent removal method for making ULVs involves solubilizing the lipids and additives with detergents by agitation or sonication to produce the desired vesicles.

Large ULVs can be prepared by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Another method of encapsulating agents in unilamellar vesicles comprises freezing/thawing an aqueous phospholipid dispersion of the agent and lipids.

In addition to the MLVs and ULVs, liposomes can also be multivesicular. These multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers.

Fatty acids (i.e., lipids) that can be conjugated to the provided compositions include those that allow the efficient incorporation of the proprotein convertase inhibitors into liposomes. Generally, the fatty acid is a polar lipid. Thus, the fatty acid can be a phospholipid The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phosphatidylglycerol (lysophosphatidylglycerol) and phosphatidic acid (lysophosphatidic acid). The monoacyl chain in these lysophosphatidyl derivatives may be palmitoyl, oleoyl, palmitoleyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

iii. In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### C. METHOD OF DECREASING VASCULAR PERMEABILITY

Provided herein is a method of decreasing vascular permeability in a blood vessel, comprising contacting the vessel with a RhoA GTPase inhibitor. Also provided is a method of decreasing vascular permeability in a blood vessel in a subject, comprising administering to the subject a RhoA GTPase inhibitor.

Vascular permeability characterizes the capacity of a blood vessel wall to pass through small molecules (ions, water, nutrients) or even whole cells (lymphocytes on their way to the site of inflammation). Blood vessel walls are lined by a single layer of endothelial cells. The gaps between endothelial cells (cell junctions) are strictly regulated depending on the type and physiological state of the tissue.

Certain growth factors, such as Vascular Permeability Factor (VPF), also known as Vascular Endothelial Growth Factor-A (VEGF) regulate vascular permeability through various transduction pathways. However, in some aspects, the herein disclosed method can decrease vascular permeability independent of these permeability factors.

Thus, also disclosed is a method of increasing endothelial cell-cell interactions, comprising contacting the endothelial cells with a RhoA GTPase inhibitor. Although not wishing to be bound by theory, in some aspects, the RhoA GTPase inhibitor of the disclosed methods modulates the actin cytoskeleton. In some aspects, the RhoA GTPase inhibitor of the disclosed methods increases pericyte and smooth muscle cell recruitment to the endothelial cells. In some aspects, the RhoA GTPase inhibitor of the disclosed methods increases the expression of, membrane localization of, or strengthening of the interactions between proteins making up the tight junctions of adjacent endothelial cells or the tight junctions within an endothelial cell. In some aspects, the RhoA GTPase inhibitor of the disclosed methods increases expression of, membrane localization of, or strengthening the interactions between proteins making up the adherens junctions of adjacent endothelial cells or the adherens junctions within an endothelial cell. In some aspects, the RhoA GTPase inhibitor of the disclosed methods modulates the interactions between the endothelium and the basement membrane. In some aspects, the RhoA GTPase inhibitor of the disclosed methods modulates the microtubule cytoskeleton. In some aspects, the RhoA GTPase inhibitor of the disclosed methods decreases vesicular transport across the endothelial cell. In some aspects, the RhoA GTPase inhibitor of the disclosed methods decreases the formation of transcellular channels (through which solutes and particles can pass).

### D. METHOD OF TREATING VASCULAR DAMAGE/DEFECTS

Provided herein is a method of treating or preventing a vascular condition in a subject, wherein the condition involves destabilization of the vascular wall, comprising administering to the subject a RhoA GTPase inhibitor. In some aspects of the method, the condition comprises edema. In some aspects of the method, the subject has not been diagnosed with a condition requiring neovascularization. In some aspects of the method, the subject is not otherwise known to be in need of a RhoA GTPase inhibitor.

Edema, formerly known as dropsy or hydropsy, is the increase of interstitial fluid in any organ—swelling. Generally, the amount of interstitial fluid is determined by the balance of fluid homeostasis, and increased secretion of fluid into the interstitium or impaired removal of this fluid may cause edema. Edema has several pathophysiologic causes, including increased hydrostatic pressure, reduced oncotic pressure, lymphatic obstruction, sodium retention, and inflammation. Edema can also be caused by defects in the vascular wall resulting in increased permeability. Disclosed herein are compositions and methods for increasing vascular permeability in the damage or defective vessels and thereby treat or prevent edema and related disorders.

1. Hemorrhage

In some aspects of the method, the subject has a vascular hemorrhage or leak. Bleeding, technically known as hemorrhage, is the loss of blood from the circulatory system. Bleeding can occur internally, where blood leaks from blood vessels inside the body or externally, either through a natural opening such as the vagina, mouth or rectum, or through a break in the skin. The complete loss of blood is referred to as exsanguination, and desanguination is a massive blood loss. Loss of 10-15% of total blood volume can be endured without clinical sequelae in a healthy person, and blood donation typically takes 8-10% of the donor's blood volume.

Hemorrhage generally becomes dangerous, or even fatal, when it causes hypovolemia (low blood volume) or hypotension (low blood pressure). In these scenarios various mechanisms come into play to maintain the body's homeostasis. These include the “retro-stress-relaxation” mechanism of cardiac muscle, the baroreceptor reflex and renal and endocrine responses such as the renin-angiotensin-aldosterone system (RAAS).

Death from hemorrhage can generally occur surprisingly quickly. This is because of ‘positive feedback’. An example of this is ‘cardiac repression’, when poor heart contraction depletes blood flow to the heart, causing even poorer heart contraction. This kind of effect causes death to occur more quickly than expected.

Hemorrhage is broken down into 4 classes by the American College of Surgeons' Advanced Trauma Life Support (ATLS). Class I Hemorrhage involves up to 15% of blood volume. There is typically no change in vital signs and fluid resuscitation is not usually necessary. Class II Hemorrhage involves 15-30% of total blood volume. A patient is often tachycardic (rapid heart beat) with a narrowing of the difference between the systolic and diastolic blood pressures. The body attempts to compensate with peripheral vasoconstriction. Skin may start to look pale and be cool to the touch. The patient might start acting differently. Volume resuscitation with crystalloids (Saline solution or Lactated Ringer's solution) is all that is typically required. Blood transfusion is not typically required. Class III Hemorrhage involves loss of 30-40% of circulating blood volume. The patient's blood pressure drops, the heart rate increases, peripheral perfusion, such as capillary refill worsens, and the mental status worsens. Fluid resuscitation with crystalloid and blood transfusion are usually necessary. Class IV Hemorrhage involves loss of >40% of circulating blood volume. The limit of the body's compensation is reached and aggressive resuscitation is required to prevent death.

2. Vascular Dysplasia

In some aspects of the method, the edema is caused by a vascular dysplasia or malformation. In some aspects of the method, the vascular dysplasia or malformation is in the brain, brain stem, or spinal cord. For example, the vascular dysplasia can be a cerebral cavernous malformation (CCM).

In some aspects, the vascular dysplasia can be caused by a genetic defect. In some aspects, the genetic defect is in a cerebral cavernous malformation (CCM) gene. For example, in some aspects, the subject has a gene mutation in CCM1, CCM2, CCM3, or a combination thereof.

In some aspects of the method, the subject has been diagnosed with a grossly dilated blood vessel. In some aspects of the method, the vascular dysplasia is a cerebral cavernous malformation. In some aspects of the method, the subject can suffer seizures or epilepsy associated with the vascular dysplasia. Thus, also disclosed is a method of treating or preventing seizures in a subject, comprising administering to the subject a RhoA GTPase inhibitor.

Cavernous angioma, also known as cerebral cavernous malformation (CCM), cavernous haemangioma, and cavernoma, is a vascular disorder of the central nervous system that may appear either sporadically or exhibit autosomal dominant inheritance. The incidence in the general population is 1%, and clinical symptoms typically appear between 20 to 30 years of age. Although these vascular lesions were once thought to be strictly congenital, they have been found to occur de novo. This disease is characterized by grossly dilated blood vessels with a single layer of endothelium and an absence of neuronal tissue within the lesions. These thinly-walled vessels resemble sinusoidal cavities filled with stagnant blood. Blood vessels in patients with CCM can range from a few millimeters to several centimeters in diameter. CCM lesions commonly resemble raspberries in external structure.

Many patients live their whole life without knowing they have a cerebral cavernous malformation. Other patients can have severe symptoms like seizures, headaches, paralysis, bleeding in the brain (cerebral hemorrhage, or hemorrhagic stroke), and even death. The nature and severity of the symptoms depend on the lesion's location in the brain. Approximately 70% of these lesions occur in the supratentorial region of the brain; the remaining 30% occur in the infratentorial region.

Clinical symptoms of this disease include recurrent headaches, focal neurological deficits, hemorrhagic stroke, and seizures, but CCM can also be asymptomatic. Diagnosis is most commonly made accidentally by routine magnetic resonance imaging (MRI) screening, but not all MRI exams are created equal. Patient can request a gradient-echo sequence in order to unmask small or punctate lesions which may otherwise remain undetected. These lesions are also more conspicuous on FLAIR imaging compared to standard T2 weighing. FLAIR imaging is different from Gradient sequences, rather, it is similar to T2 weighing but suppresses free-flowing fluid signal. Sometimes quiescent CCMs can be revealed as incidental findings during MRI exams ordered for other reasons.

Sometimes the lesion appearance imaged by MRI remains inconclusive. Consequently neurosurgeons can order a cerebral angiogram or magnetic resonance angiogram (MRA). Since CCMs are low flow lesions (they are hooked into the venous side of the circulatory system), they will be angiographically occult (invisible). If a lesion is discernible via angiogram in the same location as in the MRI, then an arteriovenous malformation (AVM) becomes the primary concern.

In up to 30% there is a coincidence of CCM with a venous angioma, also known as a developmental venous anomaly (DVA). These lesions appear either as enhancing linear blood vessels or caput medusae, a radial orientation of small vessels that resemble the hair of Medusa from Greek mythology. These lesions are thought to represent developmental anomalies of normal venous drainage. When found in association with a CCM that needs resection, great care should be taken not to disrupt the angioma.

Familial forms of CCM occur at three known genetic loci. The gene for CCM1 encodes KRIT1 (krev interaction trapped 1) and has been found to bind to ICAP1alpha (integrin cytoplasmic domain associated protein alpha), a beta1 integrin associated protein. The gene for CCM2 encodes a novel protein named “malcavernin” that contains a phosphotyrosine (PTB) binding domain. The exact biological function of CCM2 is not clear. It has been shown that CCM1 and CCM2 proteins as well as ICAP1alpha form a macromolecular complex in the cell. In addition, it appears that CCM2 protein can function as a scaffolding protein for MAP kinases that are essential in p38 activation responding to osmotic stress including MEKK3 and MKK3. It also binds to Rac and actin. Therefore, CCM2 protein is also called OSM (osmosensing scaffold for MEKK3). The CCM3 gene was the most recent CCM gene identified. CCM3 was known as PDCD10 (programmed cell death 10), which was initially identified as a gene that is up-regulated during the induction of apoptosis (cell death) in TF-1, a human myeloid cell line. PDCD10 forms complex with CCM1 protein (KRIT1) and CCM2 protein (OSM). PDCD10 interacts directly with OSM independent of KRIT1-OSM interaction.

Mutations in these three genes account for 70 to 80 percent of all cases of cerebral cavernous malformations. The remaining 20 to 30 percent of cases may be due to other, still unidentified, genes. Thus, in some aspects of the disclosed method, the subject has a gene mutation in CCM1, CCM2, CCM3, or a combination thereof.

3. Ischemia

In some aspects of the method, the edema can be caused by damage to the vascular wall. For example, the damage can be caused by ischemia. The role of ischemia on endothelial damage has been reviewed by Parolari, A., et al. (Endothelial damage during myocardial preservation and storage. 2002. Ann Thorac Surg. 73, 682-690).

In medicine, ischemia is a restriction in blood supply, generally due to factors in the blood vessels, with resultant damage or dysfunction of tissue. Rather than in hypoxia, a more general term denoting a shortage of oxygen (usually a result of lack of oxygen in the air being breathed), ischemia is an absolute or relative shortage of the blood supply to an organ. Relative shortage means the mismatch of blood supply (oxygen delivery) and blood request for adequate oxygenation of tissue.

Ischemia can also be described as an inadequate flow of blood to a part of the body, caused by constriction or blockage of the blood vessels supplying it. Ischemia of heart muscle produces angina pectoris. This can be due to Tachycardia (abnormally rapid beating of the heart); Atherosclerosis (lipid-laden plaques obstructing the lumen of arteries); Hypotension (low blood pressure, e.g. in septic shock, heart failure); Thromboembolism (blood clots); Outside compression of a blood vessel, e.g. by a tumor; Foreign bodies in the circulation (e.g. amniotic fluid in amniotic fluid embolism); Sickle cell disease (abnormally shaped hemoglobin); and Induced g-forces which restrict the blood flow and force the blood to the extremities of the body, as in aerobatics and military flying.

Since oxygen is mainly bound to hemoglobin in red blood cells, insufficient blood supply causes tissue to become hypoxic, or, if no oxygen is supplied at all, anoxic. This can cause necrosis (i.e. cell death). In very aerobic tissues such as heart and brain, at body temperature Necrosis due to ischemia usually takes about 3-4 hours before becoming irreversible. This and typically some collateral circulation to the ischemic area accounts for the efficacy of “clot-buster” drugs such as Alteplase, given for stroke and heart-attack within this time period. However, complete cessation of oxygenation of such organs for more than 20 minutes typically results in irreversible damage.

Ischemia is a feature of heart diseases, transient ischemic attacks, cerebrovascular accidents, ruptured arteriovenous malformations, and peripheral artery occlusive disease. The heart, the kidneys, and the brain are among the organs that are the most sensitive to inadequate blood supply. Ischemia in brain tissue, for example due to stroke or head injury, causes a process called the ischemic cascade to be unleashed, in which proteolytic enzymes, reactive oxygen species, and other harmful chemicals damage and may ultimately kill brain tissue.

Restoration of blood flow after a period of ischemia can actually be more damaging than the ischemia. Reintroduction of oxygen causes a greater production of damaging free radicals, resulting in reperfusion injury. With reperfusion injury, necrosis can be greatly accelerated.

4. Effect of Thrombolytic Drugs

In some aspects of the method, the damage is caused by thrombolytic drugs. Thrombolysis is the breakdown (lysis) of blood clots by pharmacological means. It is colloquially referred to as clot busting for this reason. It works by stimulating fibrinolysis by plasmin through infusion of analogs of tissue plasminogen activator, the protein that normally activates plasmin. Thrombolysis requires the use of thrombolytic drugs, which are either derived from Streptomyces spp. or the effect of recombinant technology, where human activators of plasminogen (e.g. tissue plasminogen activator, tPA) are manufactured by bacteria. Some commonly used thrombolytics are streptokinase, urokinase, alteplase (recombinant tissue plasminogen activator or rtPA), reteplase, and tenecteplase.

Formation of blood clots lies at the basis of a number of serious diseases. Diseases where thrombolysis is used include Myocardial infarction, Stroke (ischemic stroke), Massive pulmonary embolism, and Acute limb ischaemia. By breaking down the clot, the disease process can be arrested, or the complications reduced. While other anticoagulants (such as heparin) decrease the “growth” of a clot, thrombolytic agents actively reduce the size of the clot. All thrombolytic agents work by activating the enzyme plasminogen, which clears the cross-linked fibrin mesh (the backbone of a clot). This makes the clot soluble and subject to further proteolysis by other enzymes, and restores blood flow over occluded blood vessels. Apart from streptokinase, all thrombolytic drugs are administered together with heparin (unfractionated or low molecular weight heparin), usually for 24-48 hours.

Thrombolysis is usually intravenous. It can also be used during an angiogram (intra-arterial thrombolysis), e.g. when patients present with stroke beyond three hours. In some settings, emergency medical technicians can administer thrombolysis for heart attacks in prehospital settings.

These drugs are most effective if administered immediately after it has been determined they are clinically appropriate. The advantage of administration is highest within the first ninety minutes, but may extend up to six hours after the start of symptoms.

The drugs are often given in combination with intravenous heparin, or low molecular weight heparin, which are anticoagulant drugs.

Hemorrhagic stroke is a rare but serious complication of thrombolytic therapy. If a patient has had thrombolysis before, an allergy against the thrombolytic drug may have developed (especially after streptokinase). If the symptoms are mild, the infusion is stopped and the patient is commenced on an antihistamine before infusion is recommenced. Anaphylaxis generally requires immediate cessation of thrombolysis.

As disclosed herein, the use of thrombolytic drugs can substantially damage vascular endothelium, resulting in leakage, hemorrhage, and/or edema. Thus, the herein disclosed methods can be used to treat or prevent vascular damage following the use of thrombolytic drugs. In some aspects, the disclosed methods comprise administering one or more RhoA GTPase inhibitors and one or more thrombolytic drugs. The method can comprise administering the one or more RhoA GTPase inhibitors and one or more thrombolytic drugs concurrently or sequentially. Thus, also disclosed is a composition comprising one or more RhoA GTPase inhibitors and one or more thrombolytic drugs.

5. Administration

The disclosed compounds and compositions can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of the RhoA GTPase inhibitor used alone might range from about 1 μg/kg to up to 100 mg/kg of body weight or more per day, depending on the factors mentioned above.

Following administration of a disclosed composition for treating, inhibiting, or preventing edema, the efficacy of the therapeutic RhoA GTPase inhibitor can be assessed in various ways well known to the skilled practitioner.

The compositions disclosed herein may be administered prophylactically to patients or subjects who are at risk for edema or who have been or will be treated with a thrombolytic drug.

### E. SCREENING METHOD

Also provided is a method of identifying a composition that can be used to treat a vascular dysplasia, comprising contacting a cell expressing RhoA GTPase with a candidate agent, wherein a detectable decrease in RhoA GTPase levels or activity in the cell is an indication that the candidate agent can be used to treat a vascular dysplasia.

Also provided is a method of identifying a composition that can be used to treat a vascular dysplasia, comprising contacting a cell expressing ROCK1 or JNK1 with a candidate agent, wherein a detectable decrease in ROCK1 or JNK1 levels or activity in the cell is an indication that the candidate agent can be used to treat a vascular dysplasia.

Also disclosed is a method of identifying a composition that can be used to treat a vascular dysplasia, comprising providing a sample comprising RhoA under conditions that allow the binding of RhoA and ROCK1, contacting the sample with a candidate agent, detecting the level of RhoA/ROCK1 binding, comparing the binding level to a control, a decrease in RhoA/ROCK1 binding compared to the control identifying an agent that can be used to treat an inflammatory disease.

The binding of RhoA to ROCK1 can be detected using routine methods, such as immunodetection methods, that do not disturb protein binding. The levels of RhoA, ROCK1, or JNK1 can also be detected using routine methods, such as immunodetection methods. The methods can be cell-based or cell-free assays. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity on RhoA or JNK activity should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that inhibits RhoA or JNK activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Impaired Angiogenesis and Endothelial Barrier Function in a Mouse Model of Vascular Malformation

i. Results

a. Ccm2 is Required for Angiogenesis

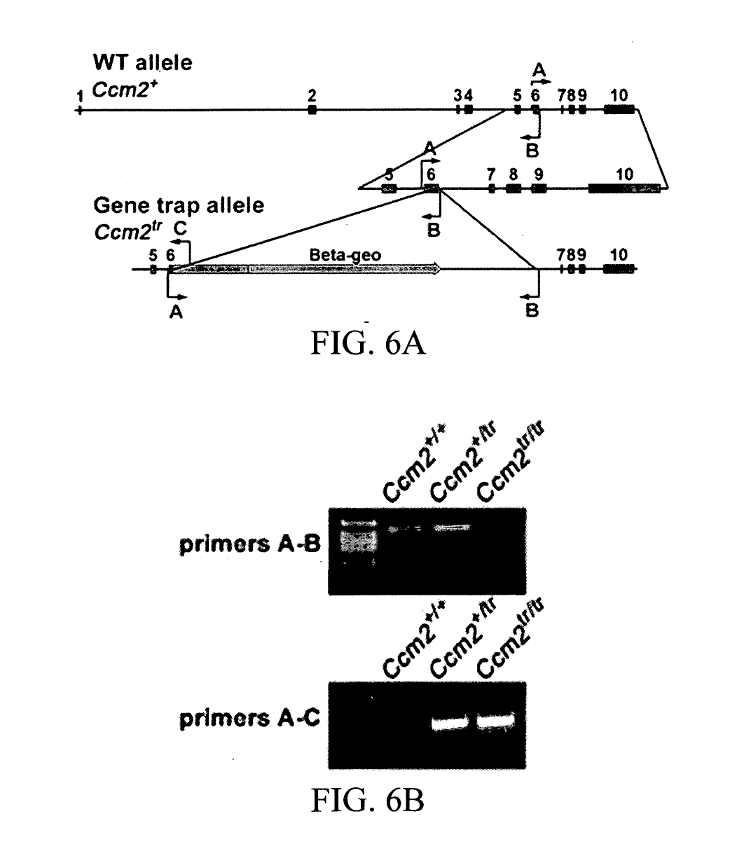

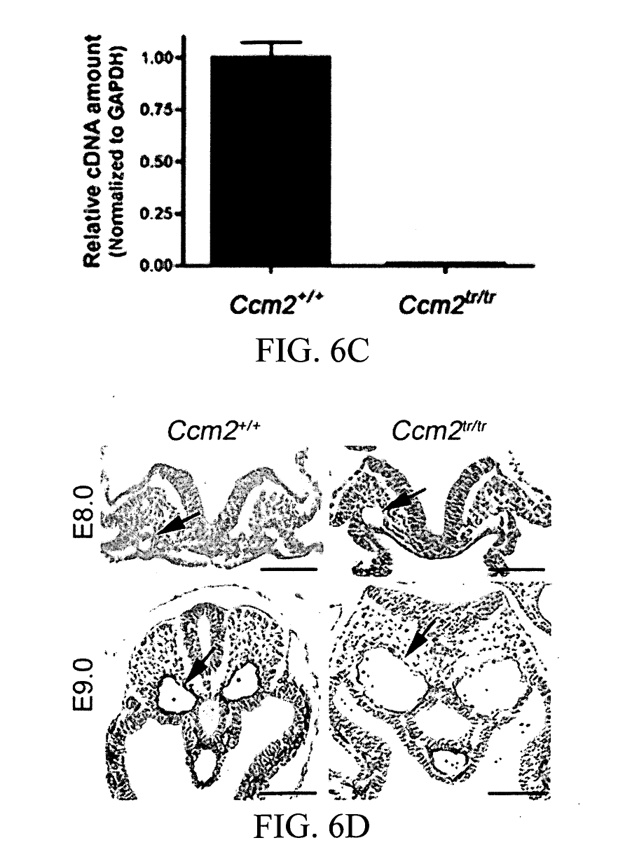

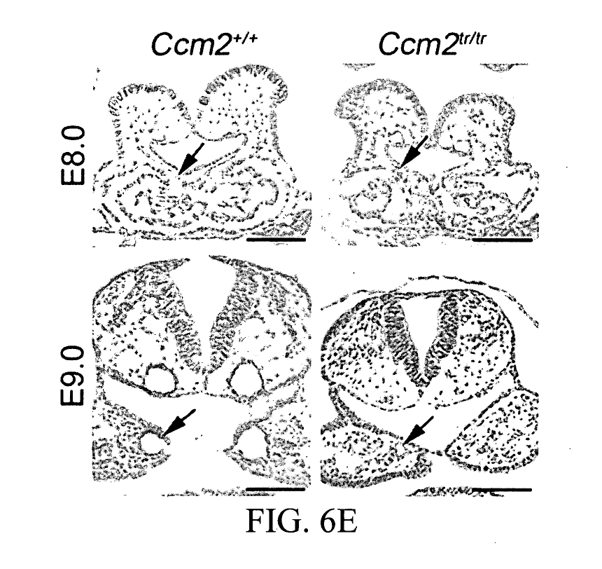

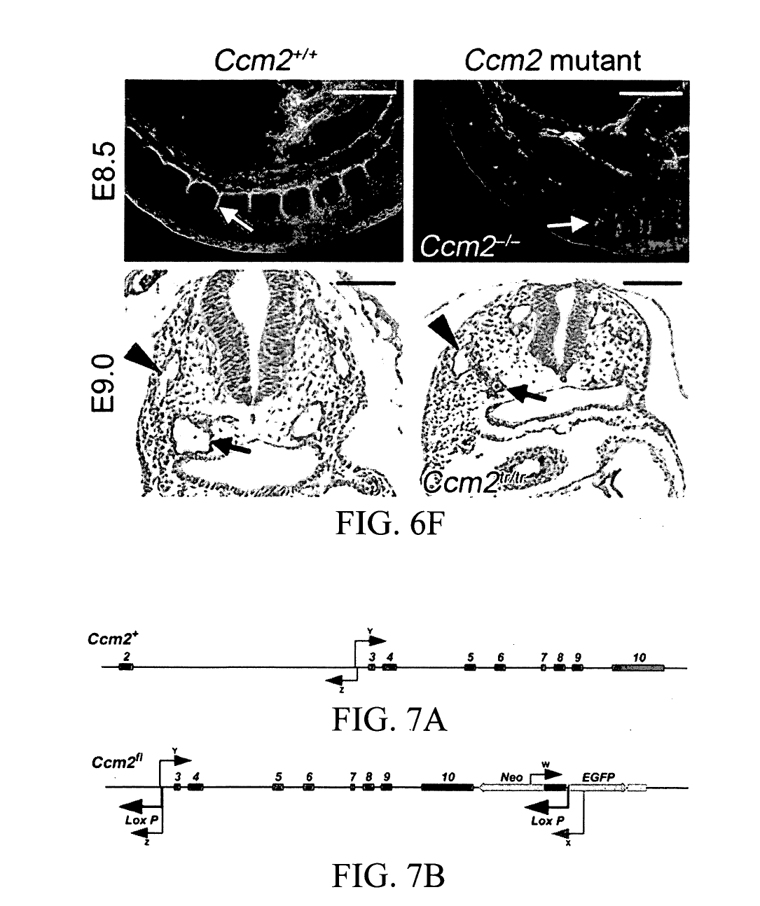

A putative null allele of Ccm2 with a gene-trap-induced mutation was identified (Plummer, N. W. et al. Neuronal expression of the Ccm2 gene in a new mouse model of cerebral cavernous malformations. 2006. Mamm. Genome 17, 119-128). This allele has been termed Ccm2^{Gt(RRG051)Byg} (hereafter designated Ccm2^{tr}), and consists of an insertion of the gene-trap vector into exon 6 of Ccm2 and a 45-nucleotide deletion of the genomic sequence (Plummer, N. W. et al. 2006), disrupting transcription of Ccm2 (FIG. 6A-C). Mice heterozygous for Ccm2^{tr} are viable and fertile as previously reported (Plummer, N. W. et al. 2006). No homozygous mutant mice were observed at weaning Mutant embryos were identified in mendelian ratios until embryonic day 9 (E9.0). Starting at E9.0, a gross phenotype was noticed in homozygous Ccm2^{tr} mice (Table 1). The homozygous mutant embryos failed to organize the yolk sac vasculature and showed evidence of growth arrest at E9.0. Pericardial effusions subsequently developed before embryo resorption at E11.5. No viable homozygous mutants were observed at E9.5 and beyond. The timing of death in these embryos is consistent with failed angiogenesis.

TABLE 1
Survival in gene trap mutants

| | Ccm2^{+/+} | Ccm2^{+/tr} | Ccm2^{tr/tr} |
|---|---|---|---|
| <E8.5 | 72 | 161 | 79 |
| E8.5 | 217 | 362 | 177 |
| E9.0 | 48 | 80 | 2 |
| >E9.0 | 79 | 121 | 0 |

Embryos from timed matings were analyzed for the appearance of a mutant phenotype. Starting at E9.0, mutant embryos could be distinguished. Homozygous mutants had abnormal yolk sac vasculature, developed growth arrest at the time of embryonic turning, and subsequently developed pericardial effusions prior to death. No viable embryos could be found at E9.5 or beyond.

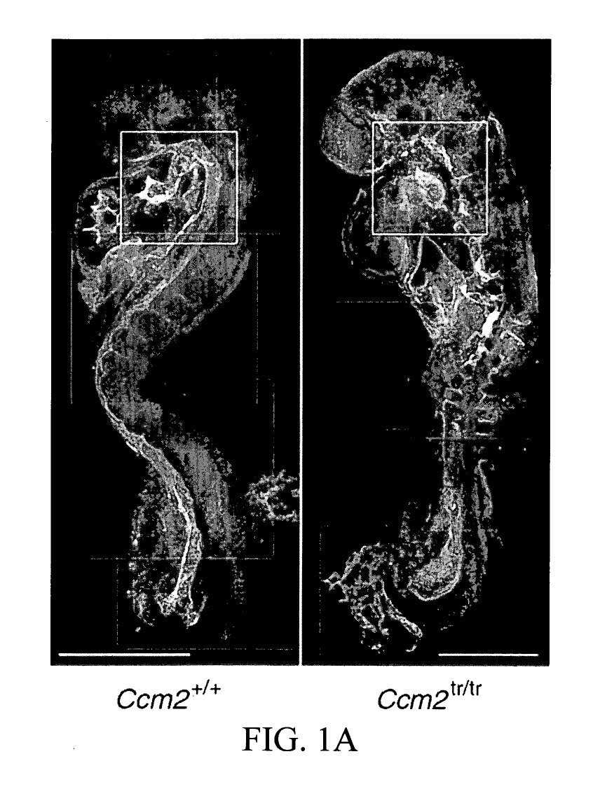

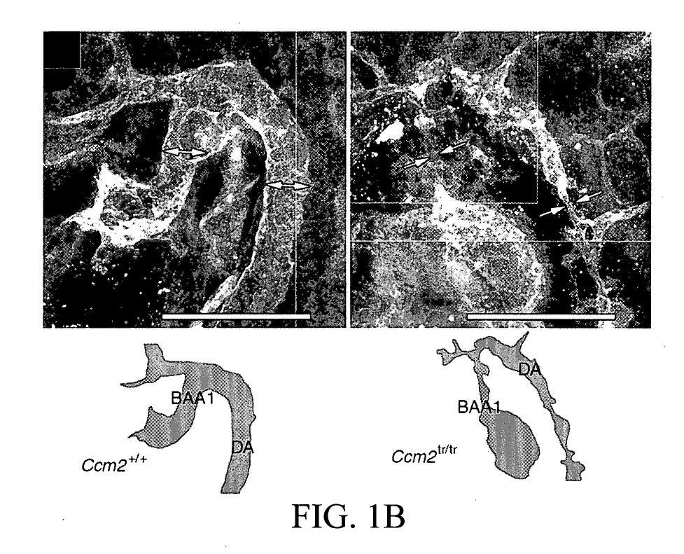

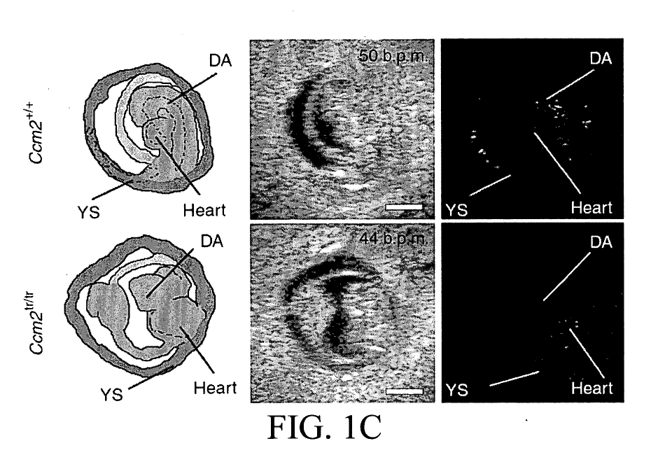

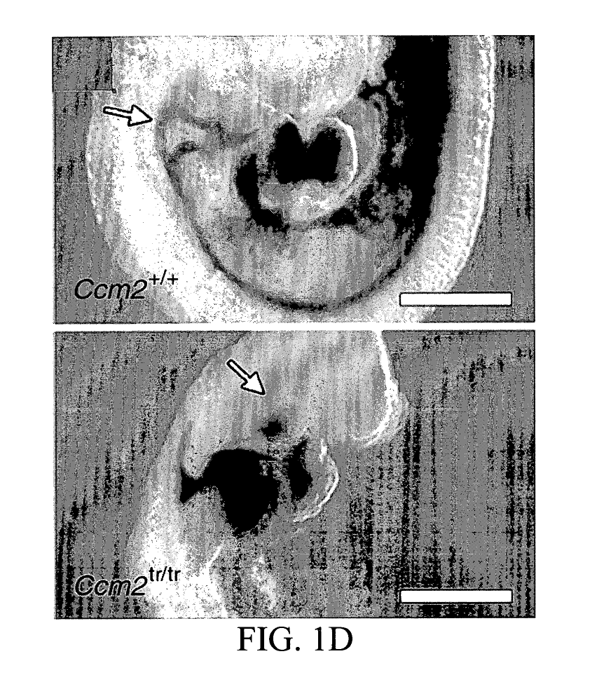

Embryos at E8.5 were studied before the mutant phenotype could be grossly detected. Embryos were stained with antibodies against the endothelial cell surface protein CD31 (PECAM) or a-smooth muscle actin and examined with whole-mount confocal immunofluorescence microscopy or sectioned and studied by immunohistochemistry. The initial patterning of the dorsal aorta (FIG. 6D,E) and yolk sac primary vascular plexus by vasculogenesis (Risau, W. Mechanisms of angiogenesis. 1997. Nature 386, 671-674) was intact in mutants. Heart development was also normal. After the initial vascular pattern was established, however, profound defects occurred in the development of subsequent vessels by angiogenesis (FIG. 1A,B). The first defects observed in mutant embryos included abnormalities of the first branchial arch artery and the intersomitic arteries at E8.5 (FIG. 1A,B and FIG. 6E,F). The first branchial arch artery, required to connect the dorsal aorta to the heart, failed to form a proper lumen. Adjacent portions of the aorta were also narrow and irregular, whereas the previously normal caudal portion of the dorsal aorta become enlarged (FIG. 6D). Yolk sac vascular remodeling was abnormal. The failure of the branchial arch arteries had profound physiologic consequences on the embryo. In vivo ultrasound studies showed that, despite normal frequency of cardiac contractions, circulation was not established in homozygous mutants (FIG. 1C). Branchial arch artery failure was not confined to the arteries of the first arch. The second and third pair of branchial arch arteries should normally form by E9.5. India ink was injected into the ventricles of mutant embryos at E9.5 and did not observe passage of ink into the dorsal aorta of mutants (FIG. 1D). Unlike the anterograde flow observed in wild-type litter-mates, ink passed retrogradely from the ventricle through the atrium and into the common cardinal vein in mutant embryos (FIG. 1D). Growth arrest and embryonic death resulted from the failed circulation first observed at E8.5.

b. Ccm2 is Required in the Endothelium

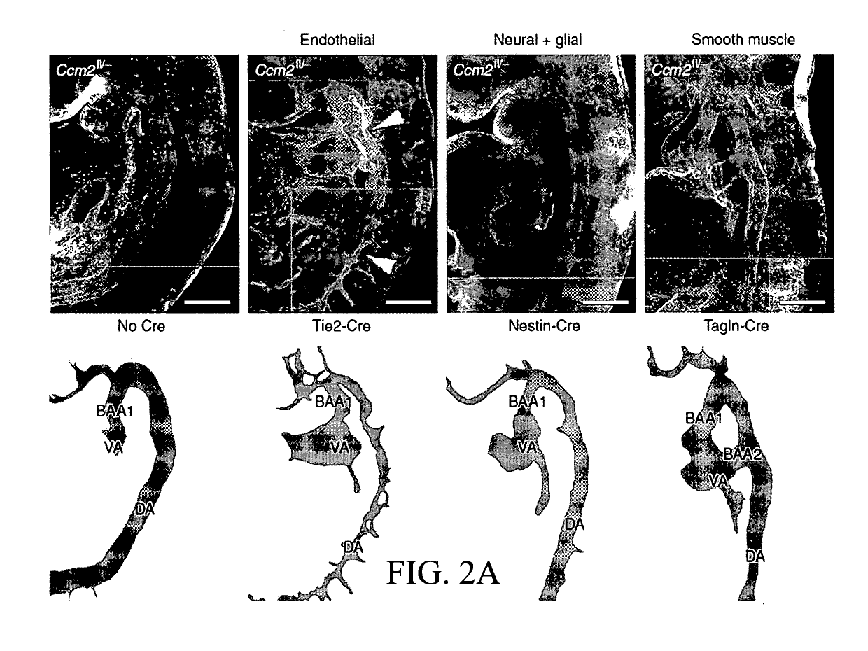

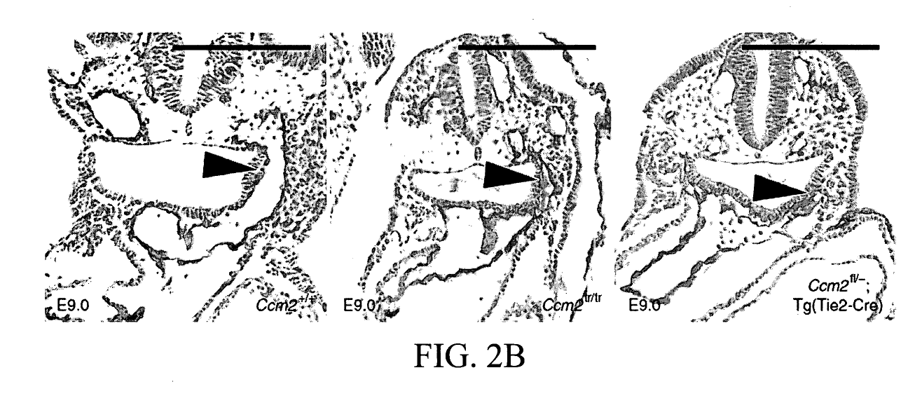

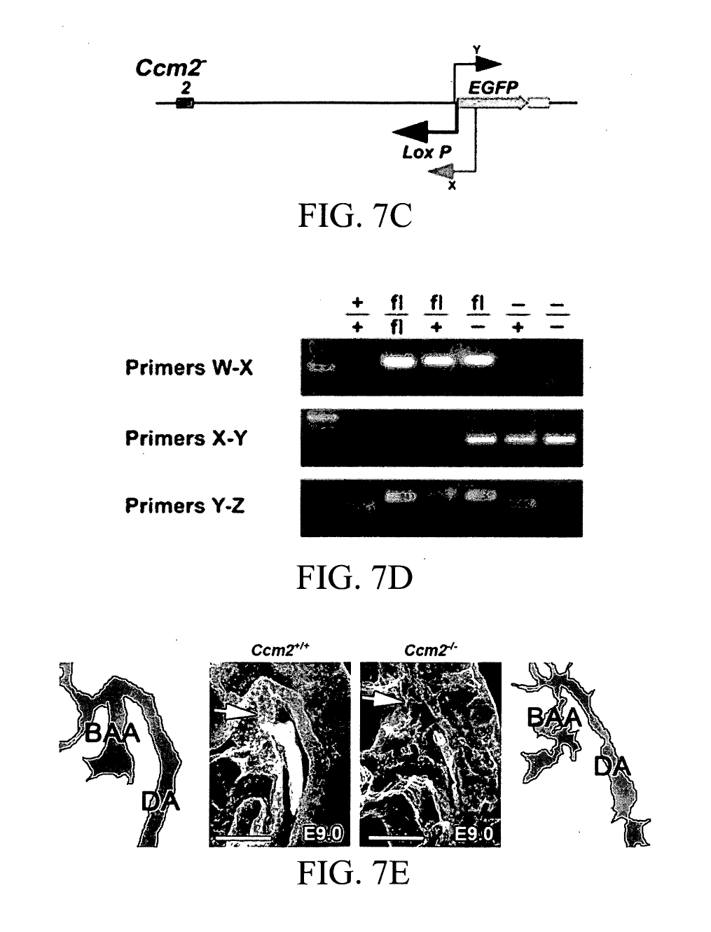

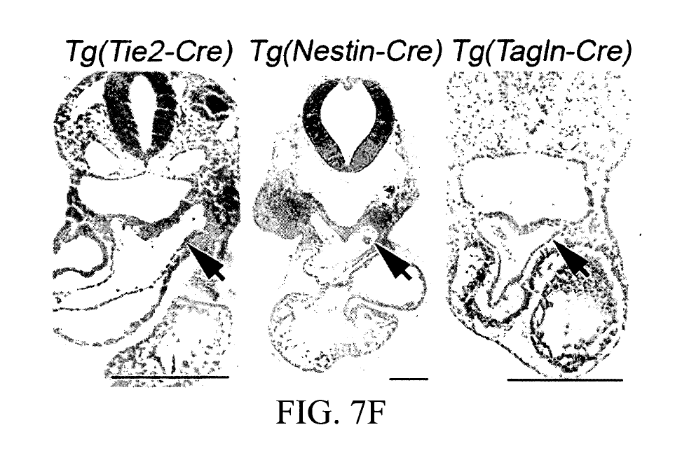

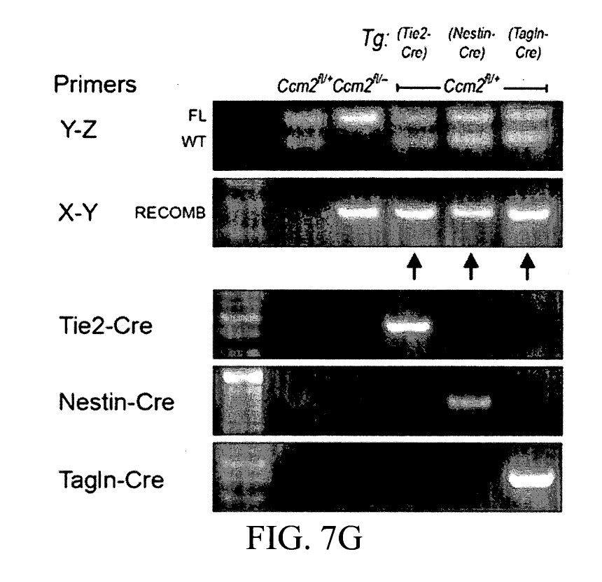

The phenotype of mice with gene-trap mutations in Ccm2 establishes an essential role for this protein in angiogenesis. This mutation is present in all cells of the embryo and thus does not make clear which tissues require Ccm2 for normal function. To determine the tissue requirement for Ccm2, mice were developed with a conditional mutation in ^{Ccm2} by Cre-lox technology (FIG. 7). This allele is termed Ccm2^{tm1Kwhi} (hereafter referred to as Ccm2^{fl}). The Ccm2 gene remains intact until the allele is exposed to Cre recombinase, which deletes exons 3-10 of Ccm2. Mating Ccm2^{fl/+} mice with HPRT-Cre mice (Su, H., Mills, A. A., Wang, X. & Bradley, A. A targeted X-linked CMV-Cre line. 2002. Genesis 32, 187-188) expressing Cre recombinase in the germline resulted in a heritable mutant allele termed Ccm2^{tm1.1Kwhi} hereafter referred to as Ccm2⁻). Homozygous Ccm2^{-/-} mutant mice phenocopy the gene-trap (Ccm2^{tr/tr}) mutants (FIG. 6E). Cre recombinase can also be expressed in a tissue-specific manner under the control of a variety of promoters. A number of tissue-restricted, somatic mutants were subsequently examined for defects in angiogenesis. Mice lacking Ccm2 in the endothelium (Ccm2⁻;Tg(Tie2-Cre))(Kisanuki, Y. Y. et al. Tie2-Cre transgenic mice: a new model for endothelial cell-lineage analysis in vivo. 2001. Dev. Biol. 230, 230-242) resemble germline mutants with similar vascular defects and timing of embryonic death (Table 2 and FIG. 2). Endothelial cell-specific deletion of Ccm2 is uniformly lethal during development (Table 2).

TABLE 2
Embryonic development in mice with tissue specific deletions of Ccm2

| | Endothelial | | | | Neural and glial | | | | Smooth muscle | | | |
|---|---|---|---|---|---|---|---|---|---|---|---|---|
| | Ccm2^{fl/+} | | Ccm2^{fl/-} | | Ccm2^{fl/+} | | Ccm2^{fl/-} | | Ccm2^{fl/+} | | Ccm2^{fl/-} | |
| | Tg | | Tg | | Tg | | Tg | | Tg | | Tg | |
| Devel. | (Tie2- | No | (Tie2- | No | (Nestin- | No | (Nestin- | No | (Tagin- | No | (Tagin- | No |
| stage | Cre) | Cre | Cre) | Cre | Cre) | Cre | Cre) | Cre | Cre) | Cre | Cre) | Cre |
| E9.0 | 4 | 12 | 13 | 9 | 6 | 7 | 7 | 5 | 6 | 4 | 6 | 2 |
| E15 | 4 | 4 | 0 | 5 | 7 | 10 | 3 | 8 | 5 | 7 | 6 | 7 |
| P0.5 | 17 | 18 | 0 | 7 | 4 | 4 | 7 | 2 | 9 | 11 | 3 | 8 |

Numbers represent the number of embryos or neonates of the indicated genotypes found at the indicated time points.

The expression of Ccm2 in neural tissues and the predominance of CCM lesions in the CNS suggest a possible role for Ccm2 in neural cells. Mice lacking Ccm2 in neural tissues were generated with Cre driven by a nestin promoter (Ccm2^{fl/-};Tg(Nes-Cre))(Sclafani, A. M. et al. Nestin-Cre mediated deletion of Pitx2 in the mouse. 2006. Genesis 44, 336-344). These mutant mice had no defects in angiogenesis at E9.0 and were found in the expected ratios at birth (Table 2 and FIG. 2). Another key contributor to the milieu of endothelial cells in vivo is the smooth muscle cell. Mice lacking Ccm2 in smooth muscle cells were generated with a transgelin-Cre (Ccm2^{fl/-};Tg(Tagln-Cre))(Lepore, J. J. et al. High-efficiency somatic mutagenesis in smooth muscle cells and cardiac myocytes in SM22-Cre transgenic mice. 2005. Genesis 41, 179-184). Like the mice lacking Ccm2 in neural tissues, mice lacking Ccm2 in smooth muscle were also found at birth, with normal vasculature at E9.0 (Table 2 and FIG. 2). These data indicate an essential role for Ccm2 only in endothelial cells for the initial events of angiogenesis.

c. CCM2 Regulates Lumen Formation Via the Actin Cytoskeleton

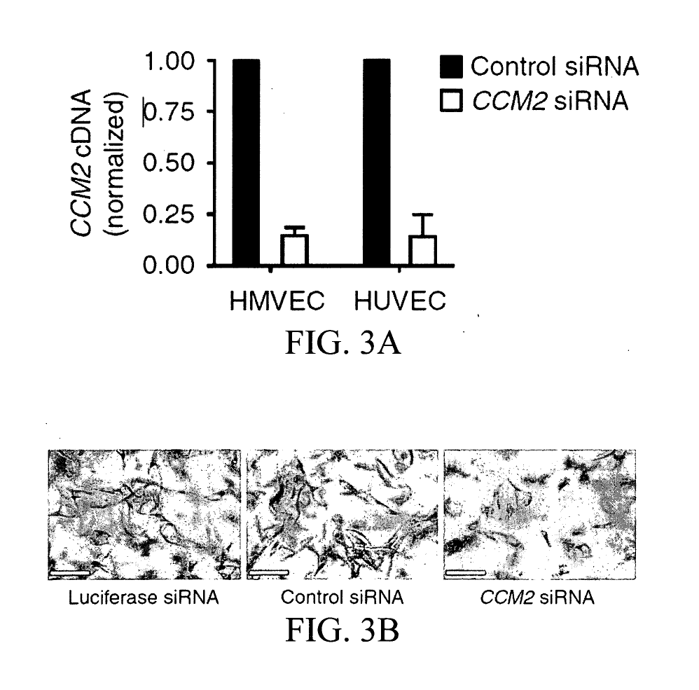

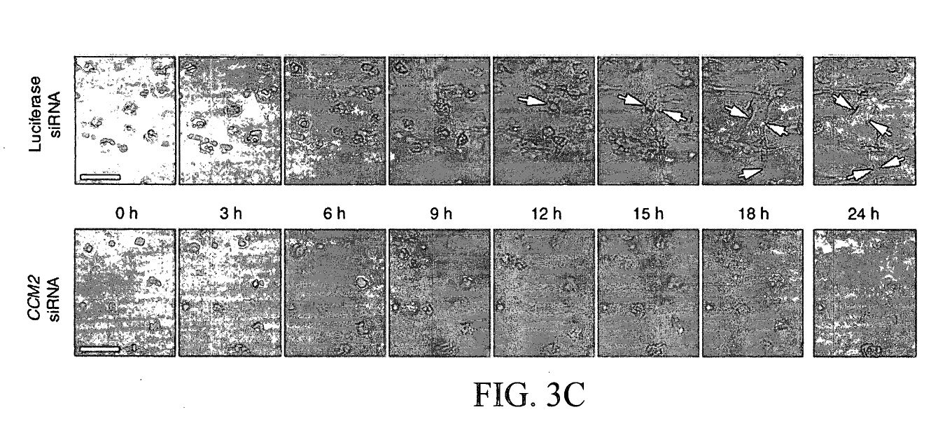

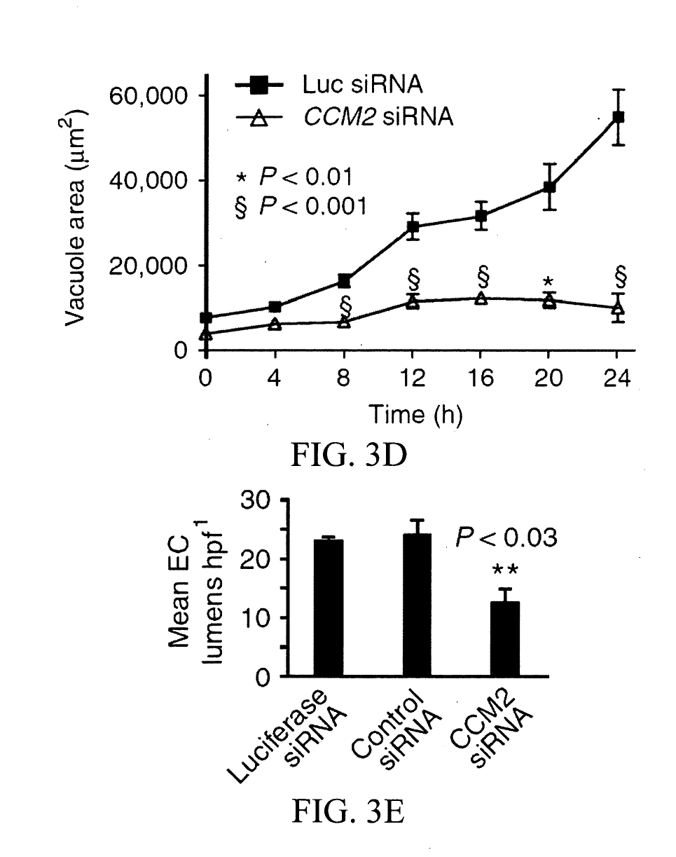

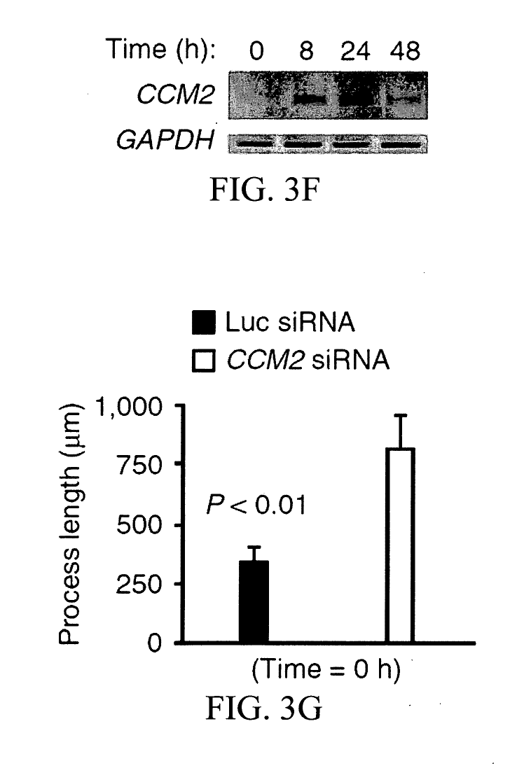

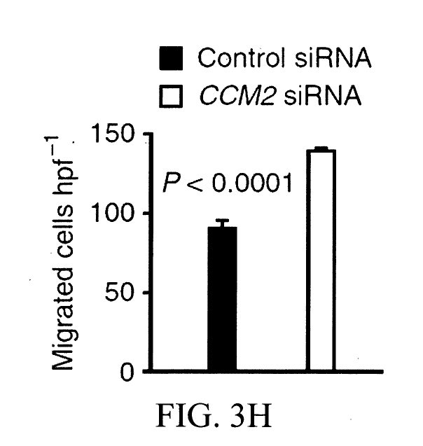

The function of CCM2 in human endothelial cells was next evaluated. CCM2 expression was detected by real-time quantitative RT-PCR in human microvascular (dermal) endothelial cells (HMVECs) and human umbilical vein endothelial cells (HUVECs; FIG. 3A). A single small interfering RNA (siRNA) construct specific for CCM2 (CCM2 siRNA) was able to decrease the amount of CCM2 transcripts by 80-90% in HMVECs and HUVECs (FIG. 3A).

Endothelial cells in three-dimensional culture spontaneously develop tube-like structures that resemble the microvasculature and that model events in developmental angiogenesis (Kamei, M. et al. Endothelial tubes assemble from intracellular vacuoles in vivo. 2006. Nature 442, 453-456). The role of CCM2 in lumen formation was tested in vitro by comparing HUVECs treated with CCM2 siRNA with either a luciferase or a random negative control siRNA in this three-dimensional assay of tube morphogenesis (FIG. 3B,C). Control HUVECs formed vacuoles that coalesced into tube-like structures over the course of 24 h, whereas CCM2-depleted HUVECs formed fewer lumens with a much smaller lumen cross-sectional area (FIG. 3D,E). This defect was observed at the single-cell stage before the formation of multicellular structures (FIG. 3D). These observations indicate a crucial and endothelial intrinsic role for CCM2 in the development of precursor vacuoles as well as in the coalescence and expansion of these structures to form the vascular lumen. Consistently, upregulation of CCM2 messenger RNA was observed by RT-PCR in a time course parallel with lumen formation in control HUVECs (FIG. 3F). The failure in lumen formation is not a consequence of insufficient endothelial migration or of an inability to form filopodial sprouts: HUVECs treated with CCM2 siRNA showed increased sprouting of cell processes when initially plated in three-dimensional culture (FIG. 3G), and HMVECs treated with CCM2 siRNA showed increased haptotactic migration (FIG. 3H).

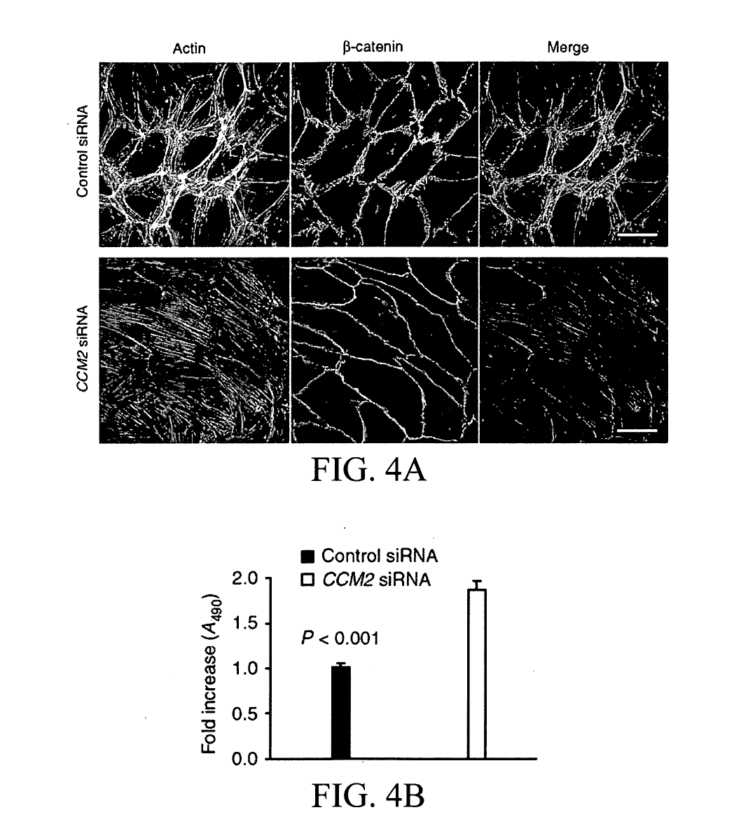

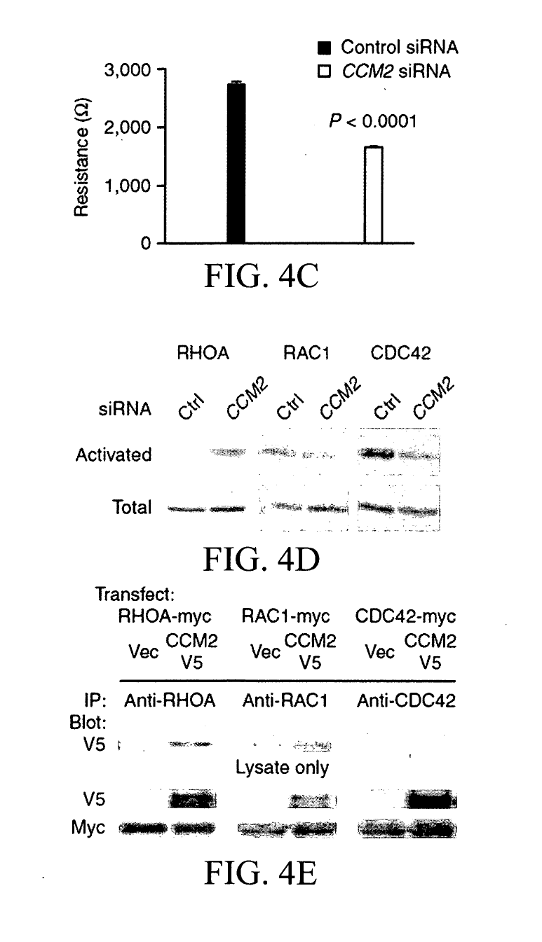

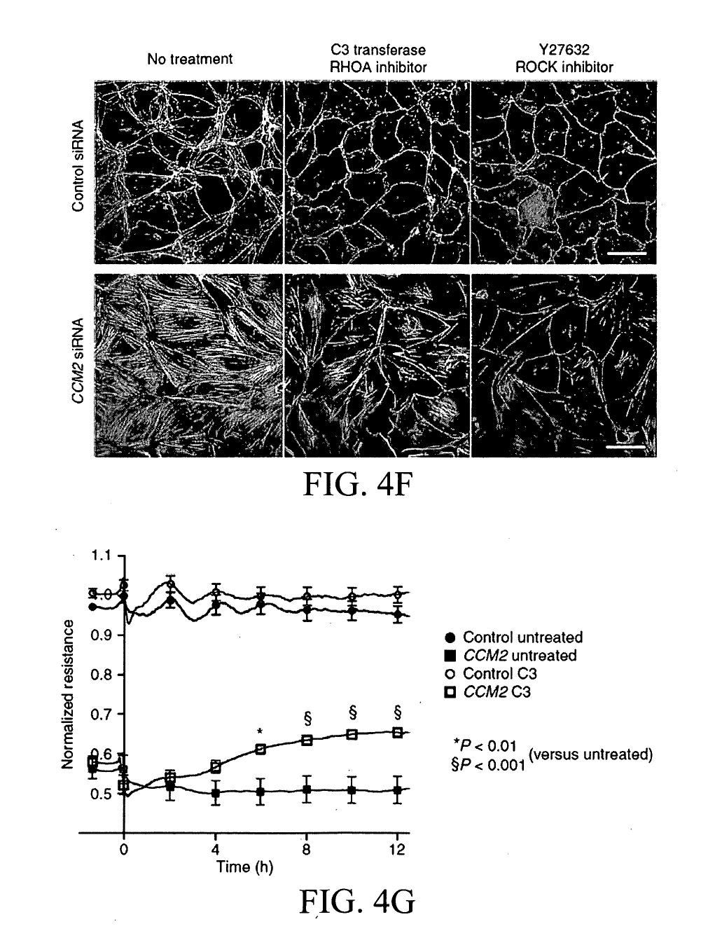

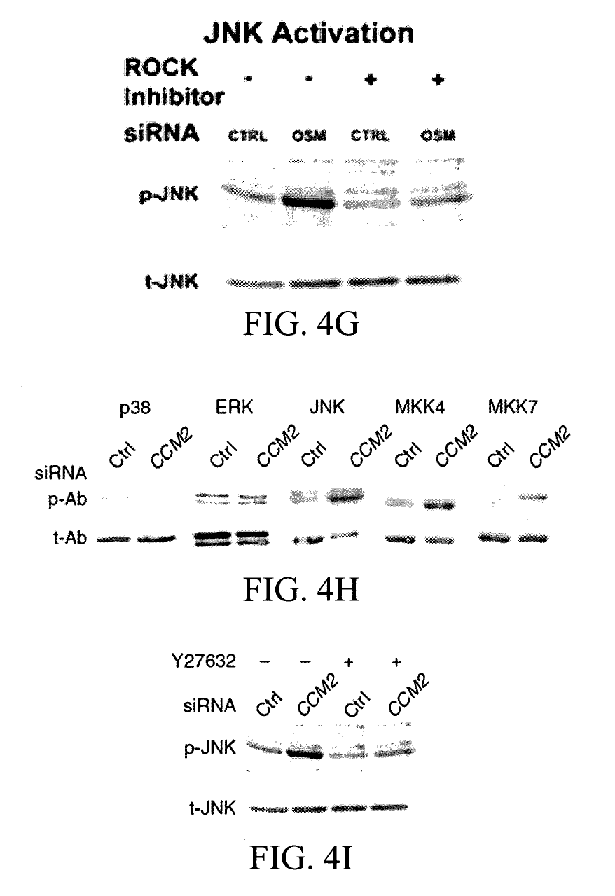

Lumen formation is dependent upon the cellular cytoskeleton (Bayless, K. J. & Davis, G. E. Microtubule depolymerization rapidly collapses capillary tube networks in vitro and angiogenic vessels in vivo through the small GTPase Rho. 2004. J. Biol. Chem. 279, 11686-11695). CCM2-deficient HMVECs had a marked increase in formation of actin stress fibers traversing the cell, with less cortical actin at the cell periphery (FIG. 4A). Actin redistribution correlated with a decrease in barrier function and increased permeability of the endothelial mono-layer (FIG. 4B,C). Decreased electrical resistance and increased transit of macromolecules (horseradish peroxidase (HRP)) was observed across CCM2-deficient monolayers compared to control cell mono-layers (FIG. 4B,C).

d. CCM2 Regulates Actin and MAPK Via RHOA

The Rho family of small GTPases regulates many aspects of the structure and function of the cellular cytoskeleton. Impaired lumen formation (Bayless, K. J. & Davis, G. E. 2004), increased formation of actin stress fibers and decreased barrier function (Wojciak-Stothard, B., Potempa, S., Eichholtz, T. & Ridley, A. J. Rho and Rac but not Cdc42 regulate endothelial cell permeability. 2001. J. Cell Sci. 114, 1343-1355) in endothelial cells suggest activation of RHOA. Consistent with the cellular phenotype, increased active (GTP-bound) RHOA was observed in CCM2-depleted HMVECs compared to control cells (FIG. 4D). No change was found in the activation of RAC1 and less basal activation of CDC42 was found (FIG. 4D). It was determined by immunoprecipitation that CCM2 binds RHOA and RAC 1 but not CDC42 (FIG. 4E). Inhibition of RHOA signaling either at the level of RHOA with C3 transferase (Mohr, C., Koch, G., Just, I. & Aktories, K. ADP-ribosylation by Clostridium botulinum C3 exoenzyme increases steady-state GTPase activities of recombinant rhoA and rhoB proteins. 1992. FEBS Lett. 297, 95-99) or downstream at the level of Rho kinase (ROCK), with the ROCK inhibitor Y-27632 (Hirose, M. et al. Molecular dissection of the Rho-associated protein kinase (p160ROCK)-regulated neurite remodeling in neuroblastoma N1E-115 cells. 1998. J. Cell Biol. 141, 1625-1636) blocked the stress fiber response of CCM2-depleted HMVECs (FIG. 4F). C3 transferase was also able to significantly rescue barrier function in these cells (FIG. 4G).

CCM2 has also been implicated in MAPK signaling (Uhlik, M. T. et al. Rac-MEKK3-MKK3 scaffolding for p38 MAPK activation during hyperosmotic shock. 2003. Nat. Cell Biol. 5, 1104-1110). Phospho-specific antibodies were used to profile the activation state of MAPK family members in the absence of CCM2 (FIG. 4H). The main families of MAP kinases are the extracellular signal regulated kinases (ERKs), p38 and JNK, with p38 and JNK also known as stress-regulated protein kinases (Kyriakis, J. M. & Avruch, J. Sounding the alarm: protein kinase cascades activated by stress and inflammation. 1996. J. Biol. Chem. 271, 24313-24316). A reduction in CCM2 transcript levels did not affect the amount of either phosphorylated ERK or phosphorylated p38 but did increase phosphorylation of JNK and its upstream kinases MKK4 and MKK7. As GTPases can stimulate MAPK signaling, it was tested whether increased JNK activation was the result of increased Rho activity by treating cells with the ROCK inhibitor Y-27632. ROCK inhibition decreased the activation of JNK (FIG. 4I). These observations indicate that the loss of CCM2 leads to RHOA activation, causing activation of JNK with an associated change in endothelial phenotype including cytoskeletal changes, impaired lumen formation and increased migration and vascular permeability.

e. Simvastatin Rescues CCM2 Deficiency In Vivo

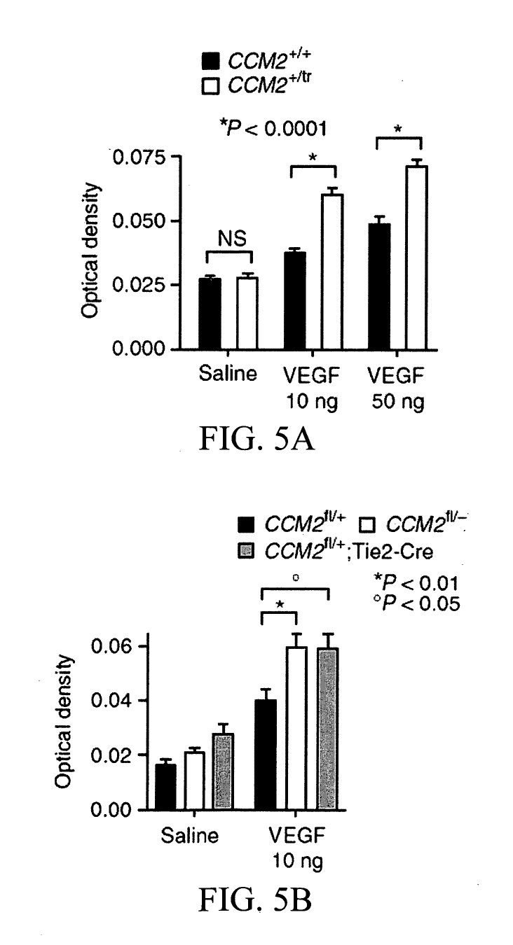

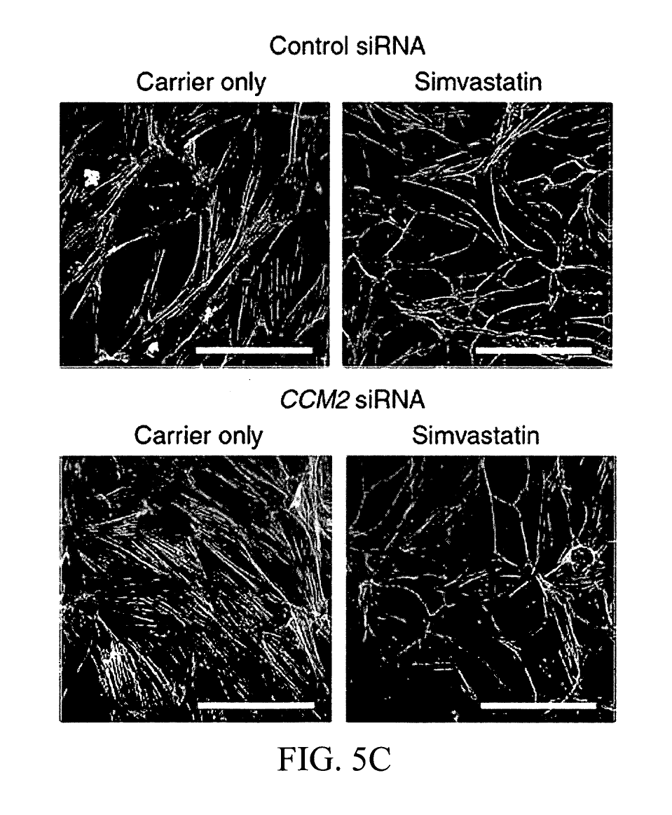

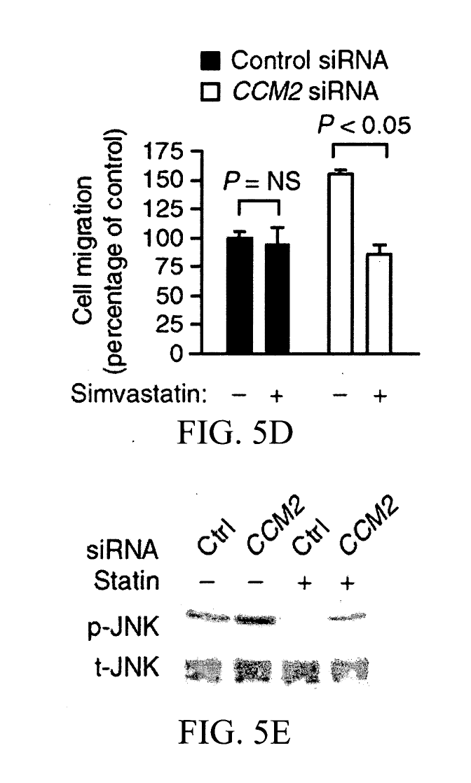

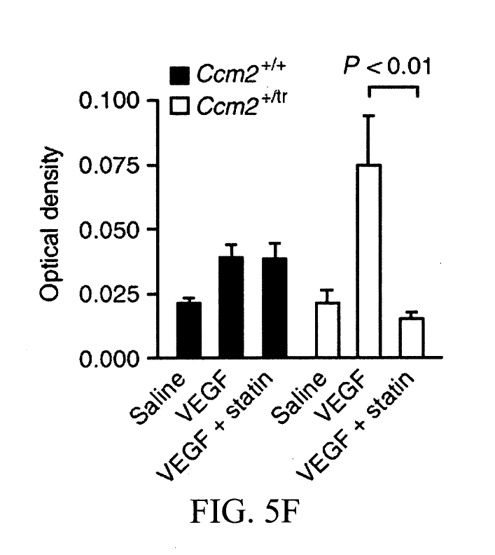

Humans with CCM have heterozygous mutations in CCM2 and suffer from vascular hemorrhage or leakage, but not from severe developmental angiogenic defects as observed in mice with homozygous mutations in Ccm2. To examine the role of Ccm2 in the disease state, attention was shifted to mice heterozygous for Ccm2. No difference was found in vascular patterning or permeability to the intravascular dye Evans blue between heterozygous mice and wild-type controls (FIG. 5A). Clinical reports indicate that physiological (Jung, K. H. et al. Cerebralcavernous malformations with dynamic and progressive course: correlation study with vascular endothelial growth factor. 2003. Arch. Neurol. 60, 1613-1618; Larson, J. J., Ball, W. S., Bove, K. E., Crone, K. R. & Tew, J. M., Jr. Formation of intracerebral cavernous malformations after radiation treatment for central nervous system neoplasia in children. 1998. J. Neurosurg. 88, 51-56; Shi, C., Shenkar, R., Batjer, H. H., Check, I. J. & Awad, I. A. Oligoclonal immune response in cerebral cavernous malformations. Laboratory investigation. 2007. J. Neurosurg. 107, 1023-1026) or genetic (Gault, J., Shenkar, R., Recksiek, P. & Awad, I. A. Biallelic somatic and germ line CCM1 truncating mutations in a cerebral cavernous malformation lesion. 2005. Stroke. 36, 872-874) stressors can have a role in disease pathogenesis. An association was observed between accelerated progression of CCM and increasing amounts of vascular endothelial growth factor (VEGF)(Jung, K. H. et al. 2003). Consistent with this clinical observation, significantly increased permeability to Evans blue was observed in Ccm2^{+/tr} mice in response to VEGF across a range of doses (FIG. 5A). Increased permeability was also observed in mice with endothelial-specific heterozygosity for Ccm2 (FIG. 5B). These results demonstrate a role for Ccm2 in the endothelium for the maintenance of normal in vivo barrier function in adults aside from its role in embryonic development.

The disclosed observations in vitro indicated that Rho inhibition can rescue the increased permeability of Ccm2-heterozygous mice. However, mice do not tolerate the ROCK inhibitor Y-27632, and the Rho inhibitor C3 transferase has poor cellular penetration, limiting its usefulness in vivo Inhibitors of 3-hydroxy-3-methyl-glutaryl-CoA reductase (statins) have pleiotropic effects that include the inhibition of Rho GTPases. Simvastatin disrupts the production of key intermediaries in the cholesterol synthesis pathway necessary for RHOA isoprenylation (Zeng, L. et al. HMG CoAreductase inhibition modulates VEGF-induced endothelial cell hyperpermeability by preventing RhoA activation and myosin regulatory light chain phosphorylation. 2005. FASEB J. 19, 1845-1847; Park, H. J. et al. 3-hydroxy-3-methylglutarylcoenzyme A reductase inhibitors interfere with angiogenesis by inhibiting the geranylgeranylation of RhoA. 2002. Circ. Res. 91, 143-150) and has been used as an inhibitor of Rho in vivo (Kranenburg, 0., Poland, M., Gebbink, M., Oomen, L. & Moolenaar, W. H. Dissociation of LPA-induced cytoskeletal contraction from stress fiber formation by differential localization of RhoA. 1997. J. Cell Sci. 110, 2417-2427; Collisson, E. A., Carranza, D. C., Chen, I. Y. & Kolodney, M. S. Isoprenylation is necessary for the full invasive potential of RhoA overexpression in human melanoma cells. 2002. J. Invest. Dermatol. 119, 1172-1176). In culture, it was determined that simvastatin reduced formation of actin stress fibers in both control and CCM2 siRNA-treated endothelial cells (FIG. 5C) and decreased haptotactic migration of CCM2-depleted HMVECs (FIG. 5D). Simvastatin also decreased the phosphorylation of JNK in both control and CCM2 siRNA-treated cells (FIG. 5E). In vivo, it was determined that pretreatment of mice with simvastatin significantly reduced the permeability response of Ccm2^{+/tr} mice to VEGF with no effect on the induced permeability of Ccm2^{+/+} mice (FIG. 5F). These data indicate that the abnormal Rho GTPase activity observed in cells depleted of CCM2 is also present in mice with reduced levels of Ccm2.

ii. Materials and Methods

Mouse strains. Mice with gene trap mutations of Ccm2 were derived from an embryonic stem cell clone (Bay Genomics). A construct for the conditional allele of Ccm2 was derived from genomic sequence obtained from a BAC clone (RP22 library, Invitrogen). The construct extended from a SalI site 5′ of exon 3 through a BamHI site 3′ of exon 10. The construct contained inserts as outlined in FIG. 7. All mice were backcrossed into the C57BL6/J strain. Experiments performed prior to the 5th cross were performed with littermate controls. LacZ reporter mice (R26R1) and Tie2-Cre mice were obtained. HPRT-Cre, Nestin-Cre and Tagln-Cre mice were obtained from The Jackson Laboratory. Genotypes were determined by PCR analysis of genomic DNA isolated from either ear biopsies or yolk sac tissues using primers outlined in FIGS. 6 and 7.

Confocal immunofluorescence of embryos. Embryos were prepared for confocal immunofluorescent detection of PECAM antigen as previously described (K. J. Whitehead, N. W. Plummer, J. A. Adams, D. A. Marchuk, D. Y. Li. 2004. Development 131:1437) with the following exception. For improved signal detection in thick specimens, following the final wash steps after applying secondary antibody, embryos were processed through graded methanols before mounting in benzyl alcohol—benzyl benzoate (BABB) based mounting medium to which similar antifade reagents had been added. Images were acquired with an Olympus FV300 confocal microscope, and stacks were chosen to visualize only one of the paired dorsal aortae. Multiple images were required to visualize the entire embryo. Photoshop® (Adobe Systems, Inc.) was used to assemble source images into a final composite (image junctions shown in final assembly).

Fetal ultrasound. Pregnant mice were studied under isoflurane anesthesia on a heated stage, with continuous monitoring of ECG and respiration. After determining pregnancy with appropriate embryonic stage, a laparotomy was performed. Embryos were studied within 45 minutes. The order of embryos was noted to correlate findings with genotype. Ultrasound images were obtained on a Vevo 660 ultrasound machine (VisualSonics) with a 40 MHz transducer. To illustrate motion in a static image, circulating blood was demonstrated by performing digital subtraction. Sequential images were applied to each other in Photoshop using a subtractive filter to remove static portions of the image. Several remaining images of moving pixels were merged together using an additive filter. This composite of dynamic pixels was colorized and overlaid upon one of the unfiltered source images.

Ink injection of embryos. Embryos were injected with India ink in the cardiac ventricle as previously described (K. J. Whitehead, N. W. Plummer, J. A. Adams, D. A. Marchuk, D. Y. Li. 2004. Development 131:1437). Embryos were studied with antibodies to Pecam (clone MEC13.3, BD Biosciences). Improved visualization on paraffin sections was obtained using a biotinylated tyramide signal amplification (TSA) kit (PerkinElmer) according to the manufacturer's instructions.

Transfection of Cells with siRNA. Human CCM2 siRNA was obtained from Dharmacon. Luciferase GL2 duplex or scramble siRNA (Dharmacon) were used as a control. EC transfection with siRNAs was carried out in growth media with 1% serum.

Immunofluorescent Cell Staining. Glass chamber slides were coated with human fibronectin (Biomedical Technologies, Inc.), and transfected cells were seeded at 50,000 cells per well. For the RhoA and ROCK experiment, cells were treated four days after seeding with 40 μg/mL of cell-permeable C3 transferase (Cytoskeleton, Inc.) or 20 μM Y-27632 for 4 hours. For the simvastatin experiment, cells were treated three days after seeding with 40 μM simvastatin (Calbiochem) or equivalent amounts of ethanol in growth media for 24 hours. Cells were fixed in 4% formaldehyde and incubated with an antibody against β-catenin (BD Biosciences). Fluorescent secondary antibody (Molecular Probes) was used to visualize β-catenin staining Actin cytoskeleton was visualized using fluorescently-conjugated phalloidin (Molecular Probes). Images were obtained with an Olympus FV300 confocal microscope.

HRP Permeability. 3 μm pore 48-well transwell inserts (Corning) were coated with human fibronectin. Transfected cells were seeded at 30,000 cells per insert. Permeability was assessed by addition of horseradish peroxidase (HRP, Sigma) to the top of the insert at a final concentration of 25 μg/mL. Solution from the bottom of the well was removed six hours later. Amount of HRP was measured using a colorimetric assay by mixing the sample with guaiacol (Sigma) and hydrogen peroxide (Fisher) and measuring the absorbance at 490 nm.

Transendothelial Resistance. An electrode culture array (Applied Biophysics) was coated with human fibronectin and transfected cells were seeded at 50,000 cells per well. Three days after seeding, cells were serum-starved in endothelial basal medium-2 with 0.2% BSA overnight. Transendothelial resistance was measured with an electric cell-substrate impedance sensing system (Applied Biophysics). Cell-permeable C3 transferase (1 μg ml⁻¹) was added to inhibit RHOA. For basal resistance, 40 wells each were measured for Rho inhibition experiments, six control wells each and ten C3 transferase wells each were measured.

MAPK Profiling. siRNA transfected cells were lysed in RIPA buffer (50 mM Tris pH 7.4, 150 mM NaCl, 1% NP-40) supplemented with protease and phosphatase inhibitors. Lysates were then analyzed by western blotting. Antibodies to phospho-JNK and total JNK were from Santa Cruz Biotechnology. Antibodies to phospho-ERK, phospho-p38, phospho-MKK4, phospho-MKK7, total ERK, total p38, total MKK4, and total MKK7 were from Cell Signaling Technology. The effect of ROCK inhibitor on JNK was tested by treating cells with 10 μM Y-27632 for 30 min prior to cell lysis. The effect of simvastatin on JNK was determined by treating cells with 10 μM simvastatin for 24 h prior to cell lysis.

Miles assay. Tail vein injections of Evans blue (0.5% in normal saline, Sigma) were performed in 8-12-week-old mice. Thirty minutes later, either saline or VEGF-165 (R&D Systems, 10 ng) was injected in multiple dermal sites. After an additional 30 min, the mice were killed, punch biopsies were performed, and Evans blue eluted from the biopsies in formamide (Invitrogen) overnight at 60° C. The absorbance of Evans blue was measured at 620 nm, subtracting the background absorbance at 740 nm. Simvastatin (20 mg kg−1) was given as an intraperitoneal injection 26 h before and 2 h before the intradermal stimuli. For the VEGF dose-response experiment, five mice were used per group. For the permeability experiment with conditional Ccm2, five Ccm2^{fl/+} mice, nine Ccm2^{fl/-} mice and ten Ccm2^{fl/+};Tg(Tie2-Cre) mice were used. For the simvastatin experiment, three mice were used with control treatment and four mice were used with simvastatin treatment.

Endothelial cell vasculogenesis in three-dimensional collagen matrices. HUVECs (passages 2-5) were suspended within 3.75 mg ml⁻¹ of collagen type I matrices and allowed to undergo morphogenesis as described (Davis, G. E. & Camarillo, C. W. An 2131 integrin-dependent pinocytic mechanism involving intracellular vacuole formation and coalescence regulates capillary lumen and tube formation in three-dimensional collagen matrix. 1996. Exp. Cell Res. 224, 39-51). Cultures were fixed with 3% glutaraldehyde for 30 min. Some cultures were stained with 0.1% toluidine blue in 30% methanol and destained before photography and visualization. Time-lapse microscopy was performed as previously described (Saunders, W. B. et al. Coregulation of vascular tube stabilization by endothelial cell TIMP-2 and pericyte TIMP-3. 2006. J. Cell Biol. 175, 179-191) with a Nikon TE2000U microscope with attached environmental chamber. Time-lapse images were examined for total area of both vacuoles and lumens (n=5 independent fields) and total process length from all cells (n=10 fields). The number of lumens per field were quantified at 24 h (n=3 fields). Metamorph (Molecular Devices) software was used to trace and quantify lumen area and process length.

Endothelial cell haptotaxis. Haptotactic migration was examined with a modified Boyden chamber assay (Neuro Probe). Polycarbonate membranes (8-μm pores) were coated with human fibronectin (1 μg ml⁻¹, Biomedical Technologies) on the lower surface. HMVECs were added to the upper well (20,000 cells per well) in endothelial growth medium-2 and allowed to migrate for 3 h. The membranes (Hema3 kit, Fisher) were fixed and stained, nonmigrated cells were removed, and the membrane mounted on a glass slide. The number of migrated cells per high-power field were counted for multiple fields and replicates (control, n=12 fields; CCM2, n=8 fields). For simvastatin rescue experiments, cells were treated with either 10 μM simvastatin (Calbiochem) or ethanol carrier for 24 h before the assay (n=3 fields per condition).

Statistical analyses. For in vitro lumen formation and cell process formation in three-dimensional culture, statistical comparisons were performed between treatment groups with a two-tailed paired samples t-test with an CL value of 0.05. For transwell in vitro permeability, transendothelial resistance, endothelial cell migration and the Miles assay of dermal permeability, group comparisons were made by two-tailed Student's t-test with an CL value of 0.05.

Digital Subtraction. Ultrasound images were selected for digital subtraction by choosing frames unaffected by motion from maternal respiration. Sequential images were applied to each other in Photoshop® (Adobe Systems, Inc.) using a subtractive filter to remove static portions of the image. Several resulting images of dynamic pixels were merged together using an additive filter. This composite of dynamic pixels was colorized using a gradient overlay. To provide some anatomic perspective, the colorized image was given 75% opacity and projected over an unfiltered source image.

Histology. Embryos were studied with antibodies to PECAM (1:250 dilution, clone MEC13.3, BD Biosciences). Improved visualization on paraffin sections was obtained using a biotinylated tyramide signal amplification (TSA) kit (PerkinElmer) according to the manufacturer's instructions. Cardiac smooth muscle was demonstrated with antibodies to alpha smooth muscle actin (1:500 dilution, clone 1A4, Sigma) without signal amplification. To demonstrate tissue specificity of transgenic Cre lines, embryos with appropriate LacZ reporter alleles were stained with X-gal.

Cell culture. Human umbilical vein endothelial cells (HUVEC) and human dermal microvascular endothelial cells (HMVEC) were obtained from Lonza and grown according to the manufacturer's instructions in EGM-2 media (HUVEC) or EGM-2MV media (HMVEC). Human embryonic kidney (HEK 293T) cells (from ATCC) were grown in Dulbecco's Modified Eagle Medium (DMEM, Gibco) with 10% fetal bovine serum (Hyclone) supplemented with antibiotics.

Reverse transcription polymerase chain reaction (RT-PCR). Total RNA was extracted from EC vasculogenesis assay at indicated time points or from siRNA-treated (Luciferase or CCM2) ECs using the ToTALLY RNA™ Isolation kit (Ambion) according to the manufacturer's instructions. RNA (1 μg) was reverse transcribed using AccuScript® High Fidelity 1st strand cDNA synthesis kit (Stratagene). For quantitative real-time PCR, total RNA was extracted from cultured endothelial cells or from embryos using the NucleoSpin® RNA II kit (Clontech) according to the manufacturer's instructions. Reverse transcription was performed with random primers using the RetroScript™ kit (Ambion). Quantitative PCR was performed with TaqMan® assays (Applied Biosystems) for human CCM2 and GAPDH, or mouse Ccm2 and Gapdh. Quantification was performed by standard curve method, and CCM2 transcripts were normalized to GAPDH for comparisons.

GTPase Activation Assays. Activity of RHOA, RAC1, and CDC42 were measured using activation assay kits (Upstate) according to manufacturer's instructions. Briefly, transfected cells were scraped into Mg²⁺ lysis buffer supplemented with protease inhibitors (Roche) and phosphatase inhibitors (Sigma). A small portion of the lysate was retained as total cell lysate and the rest was incubated with the assay reagent. GTP-bound forms were eluted from the assay reagent using Laemmli sample buffer and analyzed by western blotting. The total cell lysate was analyzed by western blotting for total GTPase input.

Immunoprecipitation. An EST for CCM2 (IMAGE: 2924210) was obtained from ATCC and cloned into a pcDNA3.1 Hygro+ plasmid modified to encode a C-terminal V5 tag. Constructs for myc-tagged RHOA, RAC1, and CDC42 were obtained from Addgene (Addgene plasmid 15899, Addgene plasmid 15902, and Addgene plasmid 15905 respectively). Plasmids were transfected into HEK 293T cells using Lipofectamine™ 2000 (Invitrogen) according to the manufacturer's instructions. 3 d post transfection, cells were scraped into lysis buffer (50 mM Tris-HCl at pH 7.5, 100 mM NaCl, 0.5% Triton X-100) supplemented with protease and phosphatase inhibitors. A portion of cell lysate was retained as whole cell lysate, and the rest incubated with antibodies against RHOA (Santa Cruz Biotechnology), RAC1, or CDC42 (Upstate) as indicated at 4° C. for 2 h, followed by incubation with Protein A/G beads (Santa Cruz Biotechnology). The beads were washed three times with lysis buffer and bound proteins were eluted using Laemmli sample buffer. Presence of CCM2-V5 was detected using an anti-V5 antibody (Invitrogen). Presence of myc-tagged RHOA, RAC1, and CDC42 were detected using an antimyc antibody (Santa Cruz Biotechnology).

Sequences. siRNA sequence used to knock down Ccm2 are as follows: sense sequence: GGAAUUGUCUCGCCAUUUAUU (SEQ ID NO: 1) and antisense sequence: 5′-UAAAUGGCGAGACAAUUCCUU (SEQ ID NO: 2), which were obtained from the company Dharmacon, part of Thermo Fisher Scientific.

Wildtype (+) allele, when differentiating from gene trap, uses primers: CCM2 WT-B: TGTAGCAATCCTCCTGCCTCTATC (SEQ ID NO: 3) and CCM2 Common D: GGTCTTCCAGATTGTTTACACGGAG (SEQ ID NO: 4).

Gene trap (tr) allele uses primers: CCM2 Common-E: TTCCAGATTGTTTACACGGAGTCC (SEQ ID NO: 5) and CCM2 KO-E2: AGGACAAGAGGGCGAGACC (SEQ ID NO: 6).

Wildtype (+) allele, when differentiating from floxed and null alleles, uses primers: CCM2-T: GACAAGGGACAGGAGCAGGC (SEQ ID NO: 7) and CCM2-U: TGGCAGGGGACAGAGTGAGG (SEQ ID NO: 8).

Floxed (fl) allele uses primers: CCM2-X: CGTAGGTCAGGGTGGTCACG (SEQ ID NO: 9) and CCM2-W: GCAATCCATCTTGTTCAATGGC (SEQ ID NO: 10).

Null (−) allele uses primers: CCM2-X: CGTAGGTCAGGGTGGTCACG (SEQ ID NO:11) and CCM2-Y: GCGTGCAAGCAAAACATCCAC (SEQ ID NO: 12).

Alternatively, the fl allele and wildtype allele will both appear, but be of different product sizes, using this pair of primers: CCM2-Y: GCGTGCAAGCAAAACATCCAC (SEQ ID NO: 13) and CCM2-Z: TGCTGAACGGTGGGCTGG (SEQ ID NO: 14).

What is claimed is:

1. A method of treating edema in a subject, comprising administering to the subject a RhoA GTPase inhibitor.

2. The method of claim 1, wherein the RhoA GTPase inhibitor is an inhibitor of 3-hydroxy-3-methylglutaryl-coenzyme A (HMG-CoA) reductase.

3. The method of claim 2, wherein the HMG CoA reductase inhibitor is a statin molecule.

4. The method of claim 3, wherein the statin molecule is Simvastatin.

5. The method of claim 1, wherein the RhoA GTPase inhibitor is an inhibitor of farnesyl diphosphate synthase (FPPS).

6. The method of claim 5, wherein the FPPS inhibitor is a nitrogen-containing bisphosphonate selected from the group consisting of Pamidronate, Neridronate, Olpadronate, Alendronate, Ibandronate, Risedronate, and Zoledronate.

7. The method of claim 1, wherein the RhoA GTPase inhibitor is an inhibitor of geranylgeranyl transferase.

8. The method of claim 7, wherein the geranylgeranyl transferase inhibitor is GGTI-2133 or GGTI-298.

9. The method of claim 1, wherein the RhoA GTPase inhibitor is an inhibitor of Rho Kinase (ROCK1).

10. The method of claim 9, wherein ROCK1 inhibitor is Y-27632, HA1077, H 1152, HA1100, or Wf-536.

11. The method of claim 1, wherein the subject has not been diagnosed with a condition requiring neovascularization.

12. The method of claim 1, wherein the edema is caused by a vascular dysplasia or malformation.

13. The method of claim 12, wherein the vascular dysplasia or malformation is in the brain, brain stem, or spinal cord.

14. The method of claim 13, wherein the vascular dysplasia is a cerebral cavernous malformation (CCM).

15. The method of claim 1, wherein the edema is caused by damage to the vascular wall.

16. The method of claim 15, wherein the damage is caused by ischemia.

17. The method of claim 15, wherein the damage is caused by thrombolytic drugs.

18. The method of claim 15, wherein the vascular dysplasia or malformation results in an increased risk of seizures, wherein the method comprises treating or preventing seizures in the subject.
